(19) United States
(12) Patent Application Publication
(10) Pub. No.: US 2024/0387006 A1
(43) Pub. Date: Nov. 21, 2024

(54) SYSTEM, METHOD, AND COMPUTER PROGRAM PRODUCT FOR PREDICTING PROPERTIES OF A POLYMER

(71) Applicant: Covestro LLC, Pittsburgh, PA (US)

(72) Inventors: James A. Thompson-Colón, Moon Township, PA (US); Timothy J. Pike, Bethel Park, PA (US); David D. Steppan, Gibsonia, PA (US); Lyubov Gindin, Pittsburgh, PA (US); Nathan Chaffin, Bethel Park, PA (US); Ronald Debiec, Pittsburgh, PA (US)

(21) Appl. No.: 18/788,331

(22) Filed: Jul. 30, 2024

Related U.S. Application Data
(63) Continuation of application No. 15/734,674, filed on Dec. 3, 2020.
(60) Provisional application No. 62/813,341, filed on Mar. 4, 2019.
(60) Provisional application No. 62/682,508, filed on Jun. 8, 2018.

(51) Int. Cl. G16C 20/30; G06N 5/04; G06N 20/00; G16C 20/70; G16C 60/00

(52) U.S. Cl. CPC G16C 20/30; G06N 5/04; G06N 20/00; G16C 20/70; G16C 60/00

(57) ABSTRACT

A system, method, and computer program product for predicting a polymer. A method may include determining one or more prediction scores for a target polymer based on at least one descriptor of the target polymer from at least three descriptor groups, wherein the one or more prediction scores include a prediction of one or more physical properties for the target polymer. A method may include determining one or more prediction scores for a target polymer based on at least one desired physical property of the target polymer, wherein the one or more prediction scores include a prediction of one or more raw materials of the target polymer.

### BACKGROUND

### Field

This disclosure relates to systems, methods, and computer programs for predicting properties of a polymer.

### Background Art

It is well understood that there are vast numbers of polymers (which for purposes of this disclosure includes polymer dispersions), including both those that are currently known and those that have yet to be discovered. Determining the properties of a polymer, whether through measurement or through some analytical method, is important in that it provides insight into how the polymer performs, and thus how the polymer can or should be used. By definition, a system is complex when behavior of the system as a whole is not derivable from properties of parts of the system: a polymer, together with its imbedded concept of molecular structure, fulfils these conditions. Molecule properties do not depend only on the properties of the component atoms but also on their mutual connections: a molecule, for example, is a holistic system, (e.g., emergent properties of the molecule cannot be derived as a sum of the properties of parts of the molecule, but the emergent properties are inherent to the whole molecule organization and stability). As a consequence of the complexity of molecular structure, molecular structure may not be represented by a unique formal model; several molecular representations can represent the same molecule, depending on the level of the underlying theoretical approach, and these representations are often not derivable from each other.

A molecular descriptor is a result of a logic and/or mathematical procedure which transforms chemical information encoded within a symbolic representation of a molecule into a useful number or a result of a standardized experiment. Molecular descriptors are applied in modelling several different properties in fields such as toxicology, analytical chemistry, physical chemistry, medicinal and pharmaceutical chemistry, environmental and toxicological studies, and regulatory tools.

### SUMMARY

According to some non-limiting embodiments or aspects, provided is a method for predicting a polymer, the method comprising: receiving, with a computer system comprising one or more processors, at least one descriptor of a target polymer from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group, wherein the atomic species count descriptor group includes one or more descriptors related to a number of at least one type of atom per an amount of the target polymer, wherein the functional count descriptor group includes one or more descriptors related to a number of at least one type of a group of atoms or bonds per the amount of the target polymer, wherein the monomer species count descriptor group includes one or more descriptors related to a number of at least one type of monomer per the amount of the target polymer, wherein the ingredient count descriptor group includes one or more descriptors related to a number of at least one type of non-monomer molecule per the amount of the target polymer, wherein the bond type count descriptor group includes one or more descriptors related to a number of at least one type of bond per the amount of the target polymer, wherein the atomic species percentage weight descriptor group includes one or more descriptors related to a percentage weight of the at least one type of atom in the target polymer, wherein the functional percentage weight descriptor group includes one or more descriptors related to a percentage weight of the at least one type of a group of atoms or bonds in the target polymer, wherein the monomer percentage weight descriptor group includes one or more descriptors related to a percentage weight of the at least one type of monomer in the target polymer, wherein the ingredient percentage weight descriptor group includes one or more descriptors related to a percentage weight of the at least one type of non-monomer molecule in the target polymer, wherein the morphology percentage weight descriptor group includes one or more descriptors related to at least one of a percentage of hard segment in the target polymer, a percentage of soft segment in the target polymer, a percentage weight of the hard segment in the target polymer, a percentage weight of the soft segment in the target polymer, or any combination thereof, wherein the polymer parameter and stoichiometry descriptor group includes one or more descriptors related to at least one ratio between the at least one type of a group of atoms or bonds and at least one prepolymer of the target polymer; determining, with the computer system, one or more prediction scores for the target polymer based on the at least one descriptor of the target polymer from the at least three of the descriptor groups and descriptor data including a plurality of descriptors of a plurality of pre-existing polymers, wherein the plurality of descriptors of the plurality of pre-existing polymers are associated with one or more physical properties of the plurality of pre-existing polymers, and wherein the one or more prediction scores include a prediction of the one or more physical properties for the target polymer; and providing, with the computer system, physical property data associated with the target polymer, wherein the physical property data is based on the one or more prediction scores.

According to some non-limiting embodiments or aspects, provided is a method for predicting a polymer, the method comprising: receiving, with a computer system comprising one or more processors, physical property data including at least one desired physical property for a target polymer; determining, with the computer system, one or more prediction scores for the target polymer based on the physical property data and at least one descriptor of a plurality of pre-existing polymers from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group; wherein the atomic species count descriptor group includes one or more descriptors related to a number of at least one type of atom per an amount of a pre-existing polymer of the plurality of pre-existing polymers, wherein the functional count descriptor group includes one or more descriptors related to a number of at least one type of a group of atoms or bonds per the amount of the pre-existing polymer, wherein the monomer species count descriptor group includes one or more descriptors related to a number of at least one type of monomer per the amount of the pre-existing polymer, wherein the ingredient count descriptor group includes one or more descriptors related to a number of at least one type of non-monomer molecule per the amount of the pre-existing polymer, wherein the bond type count descriptor group includes one or more descriptors related to a number of at least one type of bond per the amount of the pre-existing polymer, wherein the atomic species percentage weight descriptor group includes one or more descriptors related to a percentage weight of the at least one type of atom in the pre-existing polymer, wherein the functional percentage weight descriptor group includes one or more descriptors related to a percentage weight of the at least one type of a group of atoms or bonds in the pre-existing polymer, wherein the monomer percentage weight descriptor group includes one or more descriptors related to a percentage weight of the at least one type of monomer in the pre-existing polymer, wherein the ingredient percentage weight descriptor group includes one or more descriptors related to a percentage weight of the at least one type of non-monomer molecule in the pre-existing polymer, wherein the morphology percentage weight descriptor group includes one or more descriptors related to at least one of a percentage of hard segment in the pre-existing polymer, a percentage of soft segment in the pre-existing polymer, a percentage weight of the hard segment in the pre-existing polymer, a percentage weight of the soft segment in the pre-existing polymer, or any combination thereof, wherein the polymer parameter and stoichiometry descriptor group includes one or more descriptors related to at least one ratio between the at least one type of a group of atoms or bonds and at least one prepolymer of the pre-existing polymer, wherein the at least one descriptor of the plurality of pre-existing polymers from the at least three of the descriptor groups is associated with the at least one desired physical property for the plurality of pre-existing polymers, and wherein the one or more prediction scores include a prediction of one or more descriptors of the target polymer; and providing, with the computer system, a formula of the target polymer, wherein the formula of the target polymer is based on the one or more prediction scores, and wherein the formula includes a plurality of raw materials of the target polymer and percentage weights of the plurality of raw materials of the target polymer.

According to some non-limiting embodiments or aspects, provided is a computing system for predicting a polymer, comprising: one or more processors programmed or configured to: receive at least one descriptor of a target polymer from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group, wherein the atomic species count descriptor group includes one or more descriptors related to a number of at least one type of atom per an amount of the target polymer, wherein the functional count descriptor group includes one or more descriptors related to a number of at least one type of a group of atoms or bonds per the amount of the target polymer, wherein the monomer species count descriptor group includes one or more descriptors related to a number of at least one type of monomer per the amount of the target polymer, wherein the ingredient count descriptor group includes one or more descriptors related to a number of at least one type of non-monomer molecule per the amount of the target polymer, wherein the bond type count descriptor group includes one or more descriptors related to a number of at least one type of bond per the amount of the target polymer, wherein the atomic species percentage weight descriptor group includes one or more descriptors related to a percentage weight of the at least one type of atom in the target polymer, wherein the functional percentage weight descriptor group includes one or more descriptors related to a percentage weight of the at least one type of a group of atoms or bonds in the target polymer, wherein the monomer percentage weight descriptor group includes one or more descriptors related to a percentage weight of the at least one type of monomer in the target polymer, wherein the ingredient percentage weight descriptor group includes one or more descriptors related to a percentage weight of the at least one type of non-monomer molecule in the target polymer, wherein the morphology percentage weight descriptor group includes one or more descriptors related to at least one of a percentage of hard segment in the target polymer, a percentage of soft segment in the target polymer, a percentage weight of the hard segment in the target polymer, a percentage weight of the soft segment in the target polymer, or any combination thereof, wherein the polymer parameter and stoichiometry descriptor group includes one or more descriptors related to at least one ratio between the at least one type of a group of atoms or bonds and at least one prepolymer of the target polymer; determine one or more prediction scores for the target polymer based on the at least one descriptor of the target polymer from the at least three of the descriptor groups and descriptor data including a plurality of descriptors of a plurality of pre-existing polymers, wherein the plurality of descriptors of the plurality of pre-existing polymers are associated with one or more physical properties of the plurality of pre-existing polymers, and wherein the one or more prediction scores include a prediction of the one or more physical properties for the target polymer; and provide physical property data associated with the target polymer, wherein the physical property data is based on the one or more prediction scores.

According to some non-limiting embodiments or aspects, provided is a computing system for predicting a polymer, comprising: one or more processors programmed or configured to: receive physical property data including at least one desired physical property for a target polymer; determine one or more prediction scores for the target polymer based on the physical property data and at least one descriptor of a plurality of pre-existing polymers from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group; wherein the atomic species count descriptor group includes one or more descriptors related to a number of at least one type of atom per an amount of a pre-existing polymer of the plurality of pre-existing polymers, wherein the functional count descriptor group includes one or more descriptors related to a number of at least one type of a group of atoms or bonds per the amount of the pre-existing polymer, wherein the monomer species count descriptor group includes one or more descriptors related to a number of at least one type of monomer per the amount of the pre-existing polymer, wherein the ingredient count descriptor group includes one or more descriptors related to a number of at least one type of non-monomer molecule per the amount of the pre-existing polymer, wherein the bond type count descriptor group includes one or more descriptors related to a number of at least one type of bond per the amount of the pre-existing polymer, wherein the atomic species percentage weight descriptor group includes one or more descriptors related to a percentage weight of the at least one type of atom in the pre-existing polymer, wherein the functional percentage weight descriptor group includes one or more descriptors related to a percentage weight of the at least one type of a group of atoms or bonds in the pre-existing polymer, wherein the monomer percentage weight descriptor group includes one or more descriptors related to a percentage weight of the at least one type of monomer in the pre-existing polymer, wherein the ingredient percentage weight descriptor group includes one or more descriptors related to a percentage weight of the at least one type of non-monomer molecule in the pre-existing polymer, wherein the morphology percentage weight descriptor group includes one or more descriptors related to at least one of a percentage of hard segment in the pre-existing polymer, a percentage of soft segment in the pre-existing polymer, a percentage weight of the hard segment in the pre-existing polymer, a percentage weight of the soft segment in the pre-existing polymer, or any combination thereof, wherein the polymer parameter and stoichiometry descriptor group includes one or more descriptors related to at least one ratio between the at least one type of a group of atoms or bonds and at least one prepolymer of the pre-existing polymer, wherein the at least one descriptor of the plurality of pre-existing polymers from the at least three of the descriptor groups is associated with the at least one desired physical property for the plurality of pre-existing polymers, and wherein the one or more prediction scores include a prediction of one or more descriptors of the target polymer; and provide a formula of the target polymer, wherein the formula of the target polymer is based on the one or more prediction scores, and wherein the formula includes a plurality of raw materials of the target polymer and percentage weights of the plurality of raw materials of the target polymer.

According to some non-limiting embodiments or aspects, provided is a computer program product for predicting a polymer, comprising at least one non-transitory computer-readable medium including program instructions that, when executed by at least one processor cause the at least one processor to: receive at least one descriptor of a target polymer from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group, wherein the atomic species count descriptor group includes one or more descriptors related to a number of at least one type of atom per an amount of the target polymer, wherein the functional count descriptor group includes one or more descriptors related to a number of at least one type of a group of atoms or bonds per the amount of the target polymer, wherein the monomer species count descriptor group includes one or more descriptors related to a number of at least one type of monomer per the amount of the target polymer, wherein the ingredient count descriptor group includes one or more descriptors related to a number of at least one type of non-monomer molecule per the amount of the target polymer, wherein the bond type count descriptor group includes one or more descriptors related to a number of at least one type of bond per the amount of the target polymer, wherein the atomic species percentage weight descriptor group includes one or more descriptors related to a percentage weight of the at least one type of atom in the target polymer, wherein the functional percentage weight descriptor group includes one or more descriptors related to a percentage weight of the at least one type of a group of atoms or bonds in the target polymer, wherein the monomer percentage weight descriptor group includes one or more descriptors related to a percentage weight of the at least one type of monomer in the target polymer, wherein the ingredient percentage weight descriptor group includes one or more descriptors related to a percentage weight of the at least one type of non-monomer molecule in the target polymer, wherein the morphology percentage weight descriptor group includes one or more descriptors related to at least one of a percentage of hard segment in the target polymer, a percentage of soft segment in the target polymer, a percentage weight of the hard segment in the target polymer, a percentage weight of the soft segment in the target polymer, or any combination thereof, wherein the polymer parameter and stoichiometry descriptor group includes one or more descriptors related to at least one ratio between the at least one type of a group of atoms or bonds and at least one prepolymer of the target polymer; determine one or more prediction scores for the target polymer based on the at least one descriptor of the target polymer from the at least three of the descriptor groups and descriptor data including a plurality of descriptors of a plurality of pre-existing polymers, wherein the plurality of descriptors of the plurality of pre-existing polymers are associated with one or more physical properties of the plurality of pre-existing polymers, and wherein the one or more prediction scores include a prediction of the one or more physical properties for the target polymer; and provide physical property data associated with the target polymer, wherein the physical property data is based on the one or more prediction scores.

According to some non-limiting embodiments or aspects, provided is a computer program product for predicting a polymer, comprising at least one non-transitory computer-readable medium including program instructions that, when executed by at least one processor cause the at least one processor to: receive physical property data including at least one desired physical property for a target polymer; determine one or more prediction scores for the target polymer based on the physical property data and at least one descriptor of a plurality of pre-existing polymers from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group; wherein the atomic species count descriptor group includes one or more descriptors related to a number of at least one type of atom per an amount of a pre-existing polymer of the plurality of pre-existing polymers, wherein the functional count descriptor group includes one or more descriptors related to a number of at least one type of a group of atoms or bonds per the amount of the pre-existing polymer, wherein the monomer species count descriptor group includes one or more descriptors related to a number of at least one type of monomer per the amount of the pre-existing polymer, wherein the ingredient count descriptor group includes one or more descriptors related to a number of at least one type of non-monomer molecule per the amount of the pre-existing polymer, wherein the bond type count descriptor group includes one or more descriptors related to a number of at least one type of bond per the amount of the pre-existing polymer, wherein the atomic species percentage weight descriptor group includes one or more descriptors related to a percentage weight of the at least one type of atom in the pre-existing polymer, wherein the functional percentage weight descriptor group includes one or more descriptors related to a percentage weight of the at least one type of a group of atoms or bonds in the pre-existing polymer, wherein the monomer percentage weight descriptor group includes one or more descriptors related to a percentage weight of the at least one type of monomer in the pre-existing polymer, wherein the ingredient percentage weight descriptor group includes one or more descriptors related to a percentage weight of the at least one type of non-monomer molecule in the pre-existing polymer, wherein the morphology percentage weight descriptor group includes one or more descriptors related to at least one of a percentage of hard segment in the pre-existing polymer, a percentage of soft segment in the pre-existing polymer, a percentage weight of the hard segment in the pre-existing polymer, a percentage weight of the soft segment in the pre-existing polymer, or any combination thereof, wherein the polymer parameter and stoichiometry descriptor group includes one or more descriptors related to at least one ratio between the at least one type of a group of atoms or bonds and at least one prepolymer of the pre-existing polymer, wherein the at least one descriptor of the plurality of pre-existing polymers from the at least three of the descriptor groups is associated with the at least one desired physical property for the plurality of pre-existing polymers, and wherein the one or more prediction scores include a prediction of one or more descriptors of the target polymer; and provide a formula of the target polymer, wherein the formula of the target polymer is based on the one or more prediction scores, and wherein the formula includes a plurality of raw materials of the target polymer and percentage weights of the plurality of raw materials of the target polymer.

Further embodiments or aspects are set forth in the following numbered clauses:

Clause 1. A computer-implemented method for predicting a polymer, comprising: receiving, with a computer system comprising one or more processors, at least one descriptor of a target polymer from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group, wherein the atomic species count descriptor group includes one or more descriptors related to a number of at least one type of atom per an amount of the target polymer, wherein the functional count descriptor group includes one or more descriptors related to a number of at least one type of a group of atoms or bonds per the amount of the target polymer, wherein the monomer species count descriptor group includes one or more descriptors related to a number of at least one type of monomer per the amount of the target polymer, wherein the ingredient count descriptor group includes one or more descriptors related to a number of at least one type of non-monomer molecule per the amount of the target polymer, wherein the bond type count descriptor group includes one or more descriptors related to a number of at least one type of bond per the amount of the target polymer, wherein the atomic species percentage weight descriptor group includes one or more descriptors related to a percentage weight of the at least one type of atom in the target polymer, wherein the functional percentage weight descriptor group includes one or more descriptors related to a percentage weight of the at least one type of a group of atoms or bonds in the target polymer, wherein the monomer percentage weight descriptor group includes one or more descriptors related to a percentage weight of the at least one type of monomer in the target polymer, wherein the ingredient percentage weight descriptor group includes one or more descriptors related to a percentage weight of the at least one type of non-monomer molecule in the target polymer, wherein the morphology percentage weight descriptor group includes one or more descriptors related to at least one of a percentage of hard segment in the target polymer, a percentage of soft segment in the target polymer, a percentage weight of the hard segment in the target polymer, a percentage weight of the soft segment in the target polymer, or any combination thereof, wherein the polymer parameter and stoichiometry descriptor group includes one or more descriptors related to at least one ratio between the at least one type of a group of atoms or bonds and at least one prepolymer of the target polymer; determining, with the computer system, one or more prediction scores for the target polymer based on the at least one descriptor of the target polymer from the at least three of the descriptor groups and descriptor data including a plurality of descriptors of a plurality of pre-existing polymers, wherein the plurality of descriptors of the plurality of pre-existing polymers are associated with one or more physical properties of the plurality of pre-existing polymers, and wherein the one or more prediction scores include a prediction of the one or more physical properties for the target polymer; and providing, with the computer system, physical property data associated with the target polymer, wherein the physical property data is based on the one or more prediction scores.

Clause 2. The computer-implemented method of clause 1, wherein the atomic species count descriptor group includes one or more of the following descriptors of the target polymer: a number of carbon atoms per the amount of the target polymer, a number of hydrogen atoms per the amount of the target polymer, a number of oxygen atoms per the amount of the target polymer, a number of nitrogen atoms per the amount of the target polymer, and a number of sulfur atoms per the amount of the target polymer, wherein the functional count descriptor group includes one or more of the following descriptors of the target polymer: a number of carbonyl groups per the amount of the target polymer, a number of isocyanate units per the amount of the target polymer, a number of acid units per the amount of the target polymer, a number of anhydride units per the amount of the target polymer, a number of neutralizer units per the amount of the target polymer, a number of carbonate links per the amount of the target polymer, a number of ester links per the amount of the target polymer, a number of urethane links per the amount of the target polymer, a number of ether links per the amount of the target polymer, a number of hydrogen acceptors per the amount of the target polymer, a number of hydrogen donors per the amount of the target polymer, a number of methyl (CH3) groups per the amount of the target polymer, a number of methylene (CH2) groups per the amount of the target polymer, a number of methane (CH) groups per the amount of the target polymer, a number of carbon atoms without a hydrogen atom per the amount of the target polymer, a number of acrylic acid groups per the amount of the target polymer, a number of oils per the amount of the target polymer, a number of two double bonds conjugated per the amount of the target polymer, and a number of three double bonds conjugated per the amount of the target polymer, wherein the monomer species count descriptor group includes one or more of the following descriptors of the target polymer: a number of ethylene oxide groups per the amount of the target polymer, a number of propylene oxide groups per the amount of the target polymer, a number of hexanediol monomers per the amount of the target polymer, a number of butanediol monomers per the amount of the target polymer, a number of caprolactone monomers per the amount of the target polymer, a number of moles of urea per the amount of the target polymer, a number of moles of formaldehyde monomers per the amount of the target polymer, a number of dicyclohexylmethane diisocyanate monomers per the amount of the target polymer, a number of hexamethylene diisocyanate monomers per the amount of the target polymer, a number of isophorone diisocyanate monomers per the amount of the target polymer, a number of methylene diphenylmethane diisocyanate (MDI) monomers per the amount of the target polymer, a number of tolylene diisocyanate (TDI) monomers per the amount of the target polymer, a number of ethoxylated monol monomers per the amount of the target polymer, a number of propoxylated diol monomers per the amount of the target polymer, and a number of sulfonate monomers per the amount of the target polymer, wherein the ingredient count descriptor group includes one or more of the following descriptors of the target polymer: a number of polycarbonate molecules per the amount of the target polymer, a number of polyester molecules per the amount of the target polymer, a number of polyether molecules per the amount of the target polymer, a number of diamine molecules per the amount of the target polymer, a number of diacrylate ester of bisphenol A molecules per the amount of the target polymer, a number of ethanol molecules per the amount of the target polymer, a number of ethylene diamine molecules per the amount of the target polymer, a number of formaldehyde molecules per the amount of the target polymer, a number of gamma-butyrolactone molecules per the amount of the target polymer, a number of hydrazine molecules per the amount of the target polymer, a number of IPDA molecules per the amount of the target polymer, a number of isopropanol molecules per the amount of the target polymer, a number of 2-hydroxypropyl carbamate hydrazide molecules per the amount of the target polymer, a number of L-Lysin molecules per the amount of the target polymer, a number of polyester-modified acrylic oligomers per the amount of the target polymer, a number of MDI-based oligomers per the amount of the target polymer, a number of Methoxypolyethyleneglycol molecules per the amount of the target polymer, a number of Methylethylketoxime molecules per the amount of the target polymer, a number of mono-n-butylamine molecules per the amount of the target polymer, a number of monoethylene glycol molecules per the amount of the target polymer, a number of neopentylglycol molecules per the amount of the target polymer, a number of phthalic anhydride-hexane polyester molecules per the amount of the target polymer, a number of hexanediol-neopentyl glycol-adipic acid polyester molecules per the amount of the target polymer, a number of butanediol-adipic acid polyester molecules per the amount of the target polymer, a number of butanediol-neopentyl glycol-adipic acid polyester molecules per the amount of the target polymer, a number of hexanediol-adipic acid polyester molecules per the amount of the target polymer, a number of butanediol-adipic acid polyester molecules per the amount of the target polymer, a number of polyethylene glycol monomethyl ether molecules per the amount of the target polymer, a number of ethylene glycol-phthalic acid-adipic acid polyester hexanediol-isophthalic acid polyester molecules per the amount of the target polymer, and a number of ethylene glycol-phthalic acid-adipic acid polyester molecules per the amount of the target polymer, wherein the bond type count descriptor group includes one or more of the following descriptors of the target polymer: a number of linking bonds linking reactive groups per the amount of the target polymer, a number of rotating bonds linking reactive groups per the amount of the target polymer, and a number of linking bonds and non-linking bonds per the amount of the target polymer, wherein the atomic species percentage weight descriptor group includes one or more of the following descriptors of the target polymer: a percentage weight of the carbon atoms in the target polymer, a percentage weight of the hydrogen atoms in the target polymer, a percentage weight of the oxygen atoms in the target polymer, a percentage weight of the nitrogen atoms in the target polymer, and a percentage weight of the sulfur atoms in the target polymer, wherein the functional percentage weight descriptor group includes one or more of the following descriptors of the target polymer: a percentage weight of the carbonyl groups in the target polymer, a percentage weight of the isocyanate units in the target polymer, a percentage weight of the acid units in the target polymer, a percentage weight of the anhydride units in the target polymer, a percentage weight of the neutralizer units in the target polymer, a percentage weight of the carbonate links in the target polymer, a percentage weight of the ester links in the target polymer, a percentage weight of the urethane links in the target polymer, a percentage weight of the ether links in the target polymer, a percentage weight of the hydrogen acceptors in the target polymer, a percentage weight of the hydrogen donors in the target polymer, a percentage weight of the methyl (CH3) groups in the target polymer, a percentage weight of the methylene (CH2) groups in the target polymer, a percentage weight of the methane (CH) groups in the target polymer, a percentage weigh of the carbon atoms without a hydrogen atom in the target polymer, a percentage weight of the acrylic acid groups in the target polymer, a percentage weight of the oils in the target polymer, a percentage weight of the two double bonds conjugated in the target polymer, and a percentage weight of the three double bonds conjugated in the target polymer, wherein the monomer percentage weight descriptor group includes one or more of the following descriptors of the target polymer: a percentage weight of the ethylene oxide groups in the target polymer, a percentage weight of the propylene oxide groups in the target polymer, a percentage weight of the hexanediol monomers in the target polymer, a percentage weight of the butanediol monomers in the target polymer, a percentage weight of the caprolactone monomers in the target polymer, a percentage weight of the moles of urea in the target polymer, a percentage weight of the moles of formaldehyde monomers in the target polymer, a percentage weight of the dicyclohexylmethane diisocyanate monomers in the target polymer, a percentage weight of the hexamethylene diisocyanate monomers in the target polymer, a percentage weight of the isophorone diisocyanate monomers in the target polymer, a percentage weight of the MDI monomers in the target polymer, a percentage weight of the TDI monomers in the target polymer, a percentage weight of the ethoxylated monol monomers in target polymer, a percentage weight of the propoxylated diol monomers in the target polymer, and a percentage weight of the sulfonate monomers in the target polymer, wherein the ingredient percentage weight descriptor group includes one or more of the following descriptors of the target polymer: a percentage weight of the polycarbonate molecules in the target polymer, a percentage weight of the polyester molecules in the target polymer, a percentage weight of the polyether molecules in the target polymer, a percentage weight of the diamine molecules in the target polymer, a percentage weight of the diacrylate ester of bisphenol A molecules in the target polymer, a percentage weight of the ethanol molecules in the target polymer, a percentage weight of the ethylene diamine molecules in the target polymer, a percentage weight of the formaldehyde molecules in the target polymer, a percentage weight of the gamma-butyrolactone molecules in the target polymer, a percentage weight of the hydrazine molecules in the target polymer, a percentage weight of the IPDA molecules in the target polymer, a percentage weight of the isopropanol molecules in the target polymer, a percentage weight of the 2-hydroxypropyl carbamate hydrazide molecules in the target polymer, a percentage weight of the L-Lysin molecules in the target polymer, a percentage weight of the polyester-modified acrylic oligomers in the target polymer, a percentage weight of the MDI based oligomers in the target polymer, a percentage weight of the Methoxypolyethyleneglycol molecules in the target polymer, a percentage weight of the Methylethylketoxime molecules in the target polymer, a percentage weight of the mono-n-butylamine molecules in the target polymer, a percentage weight of the monoethylene glycol molecules in the target polymer, a percentage weight of the neopentylglycol molecules in the target polymer, a percentage weight of the phthalic anhydride-hexane polyester molecules in the target polymer, a percentage weight of the hexanediol-neopentyl glycol-adipic acid polyester molecules in the target polymer, a percentage weight of the butanediol-adipic acid polyester molecules in the target polymer, a percentage weight of the butanediol-neopentyl glycol-adipic acid polyester molecules in the target polymer, a percentage weight of the hexanediol-adipic acid polyester molecules in the target polymer, a percentage weight of the butanediol-adipic acid polyester molecules in the target polymer, a percentage weight of the polyethylene glycol monomethyl ether molecules in the target polymer, a percentage weight of the ethylene glycol-phthalic acid-adipic acid polyester hexanediol-isophthalic acid polyester molecules in the target polymer, and a percentage weight of the ethylene glycol-phthalic acid-adipic acid polyester molecules in the target polymer, wherein the morphology percentage weight descriptor group includes one or more of the following descriptors of the target polymer: a percentage of hard segment in the target polymer, a percentage of soft segment in the target polymer, a percentage weight of the hard segment in the target polymer, a percentage weight of the soft segment in the target polymer; and wherein the polymer parameter and stoichiometry descriptor group includes one or more of the following descriptors of the target polymer: a prepolymer number average molecular weight of the target polymer, a prepolymer weight average molecular weight of the target polymer, a prepolymer polydispersity of the target polymer, an amount of urea per the amount of the target polymer, a ratio of the amount of the urea from amine to an amount of urethane in the target polymer, a ratio of the amount of the urea from amine to an amount of urethane and urea in the target polymer, the amount of the urea and the urethane per the amount of the target polymer, a ratio of NCO equivalents to OH equivalents in the target polymer, and a ratio of NCO equivalent to NH equivalents in the target polymer.

Clause 3. The computer-implemented method of any of clauses 1 and 2, further comprising: receiving, with the computer system, a plurality of descriptors of a plurality of raw materials in the target polymer; and determining, with the computer system, the at least one descriptor of the target polymer from the at least three of the descriptor groups based on the plurality of descriptors of the plurality of raw materials.

Clause 4. The computer-implemented method of any of clauses 1-3, further comprising: determining, with the computer system, the one or more prediction scores for the target polymer based on at least one of the following additional descriptors of the target polymer: an infrared spectra of the target polymer, a Raman spectra of the target polymer, a particle size distribution of the target polymer, or any combination thereof.

Clause 5. The computer-implemented method of any of clauses 1-4, wherein the one or more physical properties of the one or more polymers of the plurality of polymers include at least one of the following: a tensile strength of the one or more polymers, an adhesion of the one or more polymers, an elongation of the one or more polymers, a hardness of the one or more polymers, a viscosity of the one or more polymers in water, or any combination thereof.

Clause 6. The computer-implemented method of any of clauses 1-5, further comprising: determining, with the computer system, the one or more prediction scores for the target polymer based on the at least one descriptor of the target polymer from at least four of the descriptor groups.

Clause 7. The computer-implemented method of any of clauses 1-6, further comprising: determining, with the computer system, the one or more prediction scores for the target polymer based on the at least one descriptor of the target polymer from at least five of the descriptor groups.

Clause 8. The computer-implemented method of any of clauses 1-7, further comprising: determining, with the computer system, the one or more prediction scores for the target polymer based on at least two descriptors of the target polymer from the at least three of the descriptor groups.

Clause 9. The computer-implemented method of any of clauses 1-8, further comprising: determining, with the computer system, the one or more prediction scores for the target polymer based on at least two descriptors of the target polymer from at least four of the descriptor groups.

Clause 10. The computer-implemented method of any of clauses 1-9, wherein the at least one descriptor of the target polymer from the at least three of the descriptor groups includes at least fifteen descriptors of the target polymer from the at least three of the descriptor groups.

Clause 11. The computer-implemented method of any of clauses 1-10, wherein the at least one descriptor of the target polymer from the at least three of the descriptor groups includes at least fifty descriptors of the target polymer from the at least three of the descriptor groups.

Clause 12. A computer-implemented method for predicting a polymer, comprising: receiving, with a computer system comprising one or more processors, physical property data including at least one desired physical property for a target polymer; determining, with the computer system, one or more prediction scores for the target polymer based on the physical property data and at least one descriptor of a plurality of pre-existing polymers from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group; wherein the atomic species count descriptor group includes one or more descriptors related to a number of at least one type of atom per an amount of a pre-existing polymer of the plurality of pre-existing polymers, wherein the functional count descriptor group includes one or more descriptors related to a number of at least one type of a group of atoms or bonds per the amount of the pre-existing polymer, wherein the monomer species count descriptor group includes one or more descriptors related to a number of at least one type of monomer per the amount of the pre-existing polymer, wherein the ingredient count descriptor group includes one or more descriptors related to a number of at least one type of non-monomer molecule per the amount of the pre-existing polymer, wherein the bond type count descriptor group includes one or more descriptors related to a number of at least one type of bond per the amount of the pre-existing polymer, wherein the atomic species percentage weight descriptor group includes one or more descriptors related to a percentage weight of the at least one type of atom in the pre-existing polymer, wherein the functional percentage weight descriptor group includes one or more descriptors related to a percentage weight of the at least one type of a group of atoms or bonds in the pre-existing polymer, wherein the monomer percentage weight descriptor group includes one or more descriptors related to a percentage weight of the at least one type of monomer in the pre-existing polymer, wherein the ingredient percentage weight descriptor group includes one or more descriptors related to a percentage weight of the at least one type of non-monomer molecule in the pre-existing polymer, wherein the morphology percentage weight descriptor group includes one or more descriptors related to at least one of a percentage of hard segment in the pre-existing polymer, a percentage of soft segment in the pre-existing polymer, a percentage weight of the hard segment in the pre-existing polymer, a percentage weight of the soft segment in the pre-existing polymer, or any combination thereof, wherein the polymer parameter and stoichiometry descriptor group includes one or more descriptors related to at least one ratio between the at least one type of a group of atoms or bonds and at least one prepolymer of the pre-existing polymer, wherein the at least one descriptor of the plurality of pre-existing polymers from the at least three of the descriptor groups is associated with the at least one desired physical property for the plurality of pre-existing polymers, and wherein the one or more prediction scores include a prediction of one or more descriptors of the target polymer; and providing, with the computer system, a formula of the target polymer, wherein the formula of the target polymer is based on the one or more prediction scores, and wherein the formula includes a plurality of raw materials of the target polymer and percentage weights of the plurality of raw materials of the target polymer.

Clause 13. The computer-implemented method of clause 12, wherein the atomic species count descriptor group includes one or more of the following descriptors of the plurality of pre-existing polymers: a number of carbon atoms per the amount of the pre-existing polymer, a number of hydrogen atoms per the amount of the pre-existing polymer, a number of oxygen atoms per the amount of the pre-existing polymer, a number of nitrogen atoms per the amount of the pre-existing polymer, and a number of sulfur atoms per the amount of the pre-existing polymer, wherein the functional count descriptor group includes one or more of the following descriptors of the plurality of pre-existing polymers: a number of carbonyl groups per the amount of the pre-existing polymer, a number of isocyanate units per the amount of the pre-existing polymer, a number of acid units per the amount of the pre-existing polymer, a number of anhydride units per the amount of the pre-existing polymer, a number of neutralizer units per the amount of the pre-existing polymer, a number of carbonate links per the amount of the pre-existing polymer, a number of ester links per the amount of the pre-existing polymer, a number of urethane links per the amount of the pre-existing polymer, a number of ether links per the amount of the pre-existing polymer, a number of hydrogen acceptors per the amount of the pre-existing polymer, a number of hydrogen donors per the amount of the pre-existing polymer, a number of methyl (CH3) groups per the amount of the pre-existing polymer, a number of methylene (CH2) groups per the amount of the pre-existing polymer, a number of methane (CH) groups per the amount of the pre-existing polymer, a number of carbon atoms without a hydrogen atom per the amount of the pre-existing polymer, a number of acrylic acid groups per the amount of the pre-existing polymer, a number of oils per the amount of the pre-existing polymer, a number of two double bonds conjugated per the amount of the pre-existing polymer, and a number of three double bonds conjugated per the amount of the pre-existing polymer, wherein the monomer species count descriptor group includes one or more of the following descriptors of the plurality of pre-existing polymers: a number of ethylene oxide groups per the amount of the pre-existing polymer, a number of propylene oxide groups per the amount of the pre-existing polymer, a number of hexanediol monomers per the amount of the pre-existing polymer, a number of butanediol monomers per the amount of the pre-existing polymer, a number of caprolactone monomers per the amount of the pre-existing polymer, a number of moles of urea per the amount of the pre-existing polymer, a number of moles of formaldehyde monomers per the amount of the pre-existing polymer, a number of dicyclohexylmethane diisocyanate monomers per the amount of the pre-existing polymer, a number of hexamethylene diisocyanate monomers per the amount of the pre-existing polymer, a number of isophorone diisocyanate monomers per the amount of the pre-existing polymer, a number of methylene diphenylmethane diisocyanate (MDI) monomers per the amount of the pre-existing polymer, a number of tolylene diisocyanate (TDI) monomers per the amount of the pre-existing polymer, a number of ethoxylated monol monomers per the amount of the pre-existing polymer, a number of propoxylated diol monomers per the amount of the pre-existing polymer, and a number of sulfonate monomers per the amount of the pre-existing polymer, wherein the ingredient count descriptor group includes one or more of the following descriptors of the plurality of pre-existing polymers: a number of polycarbonate molecules per the amount of the pre-existing polymer, a number of polyester molecules per the amount of the pre-existing polymer, a number of polyether molecules per the amount of the pre-existing polymer, a number of diamine molecules per the amount of the pre-existing polymer, a number of diacrylate ester of bisphenol A molecules per the amount of the pre-existing polymer, a number of ethanol molecules per the amount of the pre-existing polymer, a number of ethylene diamine molecules per the amount of the pre-existing polymer, a number of formaldehyde molecules per the amount of the pre-existing polymer, a number of gamma-butyrolactone molecules per the amount of the pre-existing polymer, a number of hydrazine molecules per the amount of the pre-existing polymer, a number of IPDA molecules per the amount of the pre-existing polymer, a number of isopropanol molecules per the amount of the pre-existing polymer, a number of 2-hydroxypropyl carbamate hydrazide molecules per the amount of the pre-existing polymer, a number of L-Lysin molecules per the amount of the pre-existing polymer, a number of polyester-modified acrylic oligomers per the amount of the pre-existing polymer, a number of MDI based oligomers per the amount of the pre-existing polymer, a number of Methoxypolyethyleneglycol molecules per the amount of the pre-existing polymer, a number of Methylethylketoxime molecules per the amount of the pre-existing polymer, a number of mono-n-butylamine molecules per the amount of the pre-existing polymer, a number of monoethylene glycol molecules per the amount of the pre-existing polymer, a number of neopentylglycol molecules per the amount of the pre-existing polymer, a number of phthalic anhydride-hexane polyester molecules per the amount of the pre-existing polymer, a number of hexanediol-neopentyl glycol-adipic acid polyester molecules per the amount of the pre-existing polymer, a number of butanediol-adipic acid polyester molecules per the amount of the pre-existing polymer, a number of butanediol-neopentyl glycol-adipic acid polyester molecules per the amount of the pre-existing polymer, a number of hexanediol-adipic acid polyester molecules per the amount of the pre-existing polymer, a number of butanediol-adipic acid polyester molecules per the amount of the pre-existing polymer, a number of polyethylene glycol monomethyl ether molecules per the amount of the pre-existing polymer, a number of ethylene glycol-phthalic acid-adipic acid polyester hexanediol-isophthalic acid polyester molecules per the amount of the pre-existing polymer, and a number of ethylene glycol-phthalic acid-adipic acid polyester molecules per the amount of the pre-existing polymer, wherein the bond type count descriptor group includes one or more of the following descriptors of the plurality of pre-existing polymers: a number of linking bonds linking reactive groups per the amount of the pre-existing polymer, a number of rotating bonds linking reactive groups per the amount of the pre-existing polymer, and a number of linking bonds and non-linking bonds per the amount of the pre-existing polymer, wherein the atomic species percentage weight descriptor group includes one or more of the following descriptors of the plurality of pre-existing polymers: a percentage weight of the carbon atoms in the pre-existing polymer, a percentage weight of the hydrogen atoms in the pre-existing polymer, a percentage weight of the oxygen atoms in the pre-existing polymer, a percentage weight of the nitrogen atoms in the pre-existing polymer, and a percentage weight of the sulfur atoms in the pre-existing polymer, wherein the functional percentage weight descriptor group includes one or more of the following descriptors of the plurality of pre-existing polymers: a percentage weight of the carbonyl groups in the pre-existing polymer, a percentage weight of the isocyanate units in the pre-existing polymer, a percentage weight of the acid units in the pre-existing polymer, a percentage weight of the anhydride units in the pre-existing polymer, a percentage weight of the neutralizer units in the pre-existing polymer, a percentage weight of the carbonate links in the pre-existing polymer, a percentage weight of the ester links in the pre-existing polymer, a percentage weight of the urethane links in the pre-existing polymer, a percentage weight of the ether links in the pre-existing polymer, a percentage weight of the hydrogen acceptors in the pre-existing polymer, a percentage weight of the hydrogen donors in the pre-existing polymer, a percentage weight of the methyl (CH3) groups in the pre-existing polymer, a percentage weight of the methylene (CH2) groups in the pre-existing polymer, a percentage weight of the methane (CH) groups in the pre-existing polymer, a percentage weigh of the carbon atoms without a hydrogen atom in the pre-existing polymer, a percentage weight of the acrylic acid groups in the pre-existing polymer, a percentage weight of the oils in the pre-existing polymer, a percentage weight of the two double bonds conjugated in the pre-existing polymer, and a percentage weight of the three double bonds conjugated in the pre-existing polymer, wherein the monomer percentage weight descriptor group includes one or more of the following descriptors of the plurality of pre-existing polymers: a percentage weight of the ethylene oxide groups in the pre-existing polymer, a percentage weight of the propylene oxide groups in the pre-existing polymer, a percentage weight of the hexanediol monomers in the pre-existing polymer, a percentage weight of the butanediol monomers in the pre-existing polymer, a percentage weight of the caprolactone monomers in the pre-existing polymer, a percentage weight of the moles of urea in the pre-existing polymer, a percentage weight of the moles of formaldehyde monomers in the pre-existing polymer, a percentage weight of the dicyclohexylmethane diisocyanate monomers in the pre-existing polymer, a percentage weight of the hexamethylene diisocyanate monomers in the pre-existing polymer, a percentage weight of the isophorone diisocyanate monomers in the pre-existing polymer, a percentage weight of the MDI monomers in the pre-existing polymer, a percentage weight of the TDI monomers in the pre-existing polymer, a percentage weight of the ethoxylated monol monomers in pre-existing polymer, a percentage weight of the propoxylated diol monomers in the pre-existing polymer, and a percentage weight of the sulfonate monomers in the pre-existing polymer, wherein the ingredient percentage weight descriptor group includes one or more of the following descriptors of the plurality of pre-existing polymers: a percentage weight of the polycarbonate molecules in the pre-existing polymer, a percentage weight of the polyester molecules in the pre-existing polymer, a percentage weight of the polyether molecules in the pre-existing polymer, a percentage weight of the diamine molecules in the pre-existing polymer, a percentage weight of the diacrylate ester of bisphenol A molecules in the pre-existing polymer, a percentage weight of the ethanol molecules in the pre-existing polymer, a percentage weight of the ethylene diamine molecules in the pre-existing polymer, a percentage weight of the formaldehyde molecules in the pre-existing polymer, a percentage weight of the gamma-butyrolactone molecules in the pre-existing polymer, a percentage weight of the hydrazine molecules in the pre-existing polymer, a percentage weight of the IPDA molecules in the pre-existing polymer, a percentage weight of the isopropanol molecules in the pre-existing polymer, a percentage weight of the 2-hydroxypropyl carbamate hydrazide molecules in the pre-existing polymer, a percentage weight of the L-Lysin molecules in the pre-existing polymer, a percentage weight of the polyester-modified acrylic oligomers in the pre-existing polymer, a percentage weight of the MDI based oligomers in the pre-existing polymer, a percentage weight of the Methoxypolyethyleneglycol molecules in the pre-existing polymer, a percentage weight of the Methylethylketoxime molecules in the pre-existing polymer, a percentage weight of the mono-n-butylamine molecules in the pre-existing polymer, a percentage weight of the monoethylene glycol molecules in the pre-existing polymer, a percentage weight of the neopentylglycol molecules in the pre-existing polymer, a percentage weight of the phthalic anhydride-hexane polyester molecules in the pre-existing polymer, a percentage weight of the hexanediol-neopentyl glycol-adipic acid polyester molecules in the pre-existing polymer, a percentage weight of the butanediol-adipic acid polyester molecules in the pre-existing polymer, a percentage weight of the butanediol-neopentyl glycol-adipic acid polyester molecules in the pre-existing polymer, a percentage weight of the hexanediol-adipic acid polyester molecules in the pre-existing polymer, a percentage weight of the butanediol-adipic acid polyester molecules in the pre-existing polymer, a percentage weight of the polyethylene glycol monomethyl ether molecules in the pre-existing polymer, a percentage weight of the ethylene glycol-phthalic acid-adipic acid polyester hexanediol-isophthalic acid polyester molecules in the pre-existing polymer, and a percentage weight of the ethylene glycol-phthalic acid-adipic acid polyester molecules in the pre-existing polymer, wherein the morphology percentage weight descriptor group includes one or more of the following descriptors of the plurality of pre-existing polymers: a percentage of hard segment in the pre-existing polymer, a percentage of soft segment in the pre-existing polymer, a percentage weight of the hard segment in the pre-existing polymer, a percentage weight of the soft segment in the pre-existing polymer, and wherein the polymer parameter and stoichiometry descriptor group includes one or more of the following descriptors of the plurality of pre-existing polymers: a prepolymer number average molecular weight of the pre-existing polymer, a prepolymer weight average molecular weight of the pre-existing polymer, a prepolymer polydispersity of the pre-existing polymer, an amount of urea per the amount of the pre-existing polymer, a ratio of the amount of the urea from amine to an amount of urethane in the pre-existing polymer, a ratio of the amount of the urea from amine to an amount of urethane and urea in the pre-existing polymer, the amount of the urea and the urethane per the amount of the pre-existing polymer, a ratio of NCO equivalents to OH equivalents in the pre-existing polymer, and a ratio of NCO equivalent to NH equivalents in the pre-existing polymer.

Clause 14. The computer-implemented method of any of clauses 12 and 13, wherein the at least one desired physical property includes at least one of the following: a tensile strength of the target polymer, an adhesion of the target polymer, an elongation of target polymer, a hardness of the target polymer a viscosity of the target polymer in water, or any combination thereof.

Clause 15. The computer-implemented method of any of clauses 12-14, further comprising: receiving, with the computer system, at least two desired physical properties of the target polymer, wherein a first desired physical property is assigned a first importance factor, wherein a second desired physical property is assigned a second importance factor different than the first importance factor; and determining, with the computer system, the one or more prediction scores for the target polymer based on the at least two desired physical properties of the target polymer, the first importance factor, and the second importance factor.

Clause 16. The computer-implemented method of any of clauses 12-15, further comprising: determining, with the computer system, the one or more prediction scores for the target polymer based on the at least one descriptor of the plurality of pre-existing polymers from at least four of the descriptor groups.

Clause 17. The computer-implemented method of any of clauses 12-16, further comprising: determining, with the computer system, the one or more prediction scores for the target polymer based on the at least one descriptor of the plurality of pre-existing polymers from at least five of the descriptor groups.

Clause 18. The computer-implemented method of any of clauses 12-17, further comprising: determining, with the computer system, the one or more prediction scores for the target polymer based on at least two descriptors of the plurality of pre-existing polymers from the at least three of the descriptor groups.

Clause 19. The computer-implemented method of any of clauses 12-18, further comprising: determining, with the computer system, the one or more prediction scores for the target polymer based on at least two descriptors of the plurality of pre-existing polymers from at least four of the descriptor groups.

Clause 20. The computer-implemented method of any of clauses 12-19, wherein the at least one descriptor of the plurality of pre-existing polymers from the at least three of the descriptor groups includes at least fifteen descriptors of the plurality of pre-existing polymers from the at least three of the descriptor groups.

Clause 21. The computer-implemented method of any of clauses 12-20, wherein the at least one descriptor of the plurality of pre-existing polymers from the at least three of the descriptor groups includes at least fifty descriptors of the plurality of pre-existing polymers from the at least three of the descriptor groups.

Clause 22. A computing system for predicting a polymer, comprising: one or more processors programmed or configured to: receive at least one descriptor of a target polymer from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group, wherein the atomic species count descriptor group includes one or more descriptors related to a number of at least one type of atom per an amount of the target polymer, wherein the functional count descriptor group includes one or more descriptors related to a number of at least one type of a group of atoms or bonds per the amount of the target polymer, wherein the monomer species count descriptor group includes one or more descriptors related to a number of at least one type of monomer per the amount of the target polymer, wherein the ingredient count descriptor group includes one or more descriptors related to a number of at least one type of non-monomer molecule per the amount of the target polymer, wherein the bond type count descriptor group includes one or more descriptors related to a number of at least one type of bond per the amount of the target polymer, wherein the atomic species percentage weight descriptor group includes one or more descriptors related to a percentage weight of the at least one type of atom in the target polymer, wherein the functional percentage weight descriptor group includes one or more descriptors related to a percentage weight of the at least one type of a group of atoms or bonds in the target polymer, wherein the monomer percentage weight descriptor group includes one or more descriptors related to a percentage weight of the at least one type of monomer in the target polymer, wherein the ingredient percentage weight descriptor group includes one or more descriptors related to a percentage weight of the at least one type of non-monomer molecule in the target polymer, wherein the morphology percentage weight descriptor group includes one or more descriptors related to at least one of a percentage of hard segment in the target polymer, a percentage of soft segment in the target polymer, a percentage weight of the hard segment in the target polymer, a percentage weight of the soft segment in the target polymer, or any combination thereof, wherein the polymer parameter and stoichiometry descriptor group includes one or more descriptors related to at least one ratio between the at least one type of a group of atoms or bonds and at least one prepolymer of the target polymer; determine one or more prediction scores for the target polymer based on the at least one descriptor of the target polymer from the at least three of the descriptor groups and descriptor data including a plurality of descriptors of a plurality of pre-existing polymers, wherein the plurality of descriptors of the plurality of pre-existing polymers are associated with one or more physical properties of the plurality of pre-existing polymers, and wherein the one or more prediction scores include a prediction of the one or more physical properties for the target polymer; and provide physical property data associated with the target polymer, wherein the physical property data is based on the one or more prediction scores.

Clause 23. A computing system for predicting a polymer, comprising: one or more processors programmed or configured to: receive physical property data including at least one desired physical property for a target polymer; determine one or more prediction scores for the target polymer based on the physical property data and at least one descriptor of a plurality of pre-existing polymers from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group; wherein the atomic species count descriptor group includes one or more descriptors related to a number of at least one type of atom per an amount of a pre-existing polymer of the plurality of pre-existing polymers, wherein the functional count descriptor group includes one or more descriptors related to a number of at least one type of a group of atoms or bonds per the amount of the pre-existing polymer, wherein the monomer species count descriptor group includes one or more descriptors related to a number of at least one type of monomer per the amount of the pre-existing polymer, wherein the ingredient count descriptor group includes one or more descriptors related to a number of at least one type of non-monomer molecule per the amount of the pre-existing polymer, wherein the bond type count descriptor group includes one or more descriptors related to a number of at least one type of bond per the amount of the pre-existing polymer, wherein the atomic species percentage weight descriptor group includes one or more descriptors related to a percentage weight of the at least one type of atom in the pre-existing polymer, wherein the functional percentage weight descriptor group includes one or more descriptors related to a percentage weight of the at least one type of a group of atoms or bonds in the pre-existing polymer, wherein the monomer percentage weight descriptor group includes one or more descriptors related to a percentage weight of the at least one type of monomer in the pre-existing polymer, wherein the ingredient percentage weight descriptor group includes one or more descriptors related to a percentage weight of the at least one type of non-monomer molecule in the pre-existing polymer, wherein the morphology percentage weight descriptor group includes one or more descriptors related to at least one of a percentage of hard segment in the pre-existing polymer, a percentage of soft segment in the pre-existing polymer, a percentage weight of the hard segment in the pre-existing polymer, a percentage weight of the soft segment in the pre-existing polymer, or any combination thereof, wherein the polymer parameter and stoichiometry descriptor group one or more descriptors related to includes at least one ratio between the at least one type of a group of atoms or bonds and at least one prepolymer of the pre-existing polymer, wherein the at least one descriptor of the plurality of pre-existing polymers from the at least three of the descriptor groups is associated with the at least one desired physical property for the plurality of pre-existing polymers, and wherein the one or more prediction scores include a prediction of one or more descriptors of the target polymer; and provide a formula of the target polymer, wherein the formula of the target polymer is based on the one or more prediction scores, and wherein the formula includes a plurality of raw materials of the target polymer and percentage weights of the plurality of raw materials of the target polymer.

Clause 24. A computer program product for predicting a polymer, comprising at least one non-transitory computer-readable medium including program instructions that, when executed by at least one processor cause the at least one processor to: receive at least one descriptor of a target polymer from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group, wherein the atomic species count descriptor group includes one or more descriptors related to a number of at least one type of atom per an amount of the target polymer, wherein the functional count descriptor group includes one or more descriptors related to a number of at least one type of a group of atoms or bonds per the amount of the target polymer, wherein the monomer species count descriptor group includes one or more descriptors related to a number of at least one type of monomer per the amount of the target polymer, wherein the ingredient count descriptor group includes one or more descriptors related to a number of at least one type of non-monomer molecule per the amount of the target polymer, wherein the bond type count descriptor group includes one or more descriptors related to a number of at least one type of bond per the amount of the target polymer, wherein the atomic species percentage weight descriptor group includes one or more descriptors related to a percentage weight of the at least one type of atom in the target polymer, wherein the functional percentage weight descriptor group includes one or more descriptors related to a percentage weight of the at least one type of a group of atoms or bonds in the target polymer, wherein the monomer percentage weight descriptor group includes one or more descriptors related to a percentage weight of the at least one type of monomer in the target polymer, wherein the ingredient percentage weight descriptor group includes one or more descriptors related to a percentage weight of the at least one type of non-monomer molecule in the target polymer, wherein the morphology percentage weight descriptor group includes one or more descriptors related to at least one of a percentage of hard segment in the target polymer, a percentage of soft segment in the target polymer, a percentage weight of the hard segment in the target polymer, a percentage weight of the soft segment in the target polymer, or any combination thereof, wherein the polymer parameter and stoichiometry descriptor group includes one or more descriptors related to at least one ratio between the at least one type of a group of atoms or bonds and at least one prepolymer of the target polymer; determine one or more prediction scores for the target polymer based on the at least one descriptor of the target polymer from the at least three of the descriptor groups and descriptor data including a plurality of descriptors of a plurality of pre-existing polymers, wherein the plurality of descriptors of the plurality of pre-existing polymers are associated with one or more physical properties of the plurality of pre-existing polymers, and wherein the one or more prediction scores include a prediction of the one or more physical properties for the target polymer; and provide physical property data associated with the target polymer, wherein the physical property data is based on the one or more prediction scores.

Clause 25. A computer program product for predicting a polymer, comprising at least one non-transitory computer-readable medium including program instructions that, when executed by at least one processor cause the at least one processor to: receive physical property data including at least one desired physical property for a target polymer; determine one or more prediction scores for the target polymer based on the physical property data and at least one descriptor of a plurality of pre-existing polymers from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group; wherein the atomic species count descriptor group includes one or more descriptors related to a number of at least one type of atom per an amount of a pre-existing polymer of the plurality of pre-existing polymers, wherein the functional count descriptor group includes one or more descriptors related to a number of at least one type of a group of atoms or bonds per the amount of the pre-existing polymer, wherein the monomer species count descriptor group includes one or more descriptors related to a number of at least one type of monomer per the amount of the pre-existing polymer, wherein the ingredient count descriptor group includes one or more descriptors related to a number of at least one type of non-monomer molecule per the amount of the pre-existing polymer, wherein the bond type count descriptor group includes one or more descriptors related to a number of at least one type of bond per the amount of the pre-existing polymer, wherein the atomic species percentage weight descriptor group includes one or more descriptors related to a percentage weight of the at least one type of atom in the pre-existing polymer, wherein the functional percentage weight descriptor group includes one or more descriptors related to a percentage weight of the at least one type of a group of atoms or bonds in the pre-existing polymer, wherein the monomer percentage weight descriptor group includes one or more descriptors related to a percentage weight of the at least one type of monomer in the pre-existing polymer, wherein the ingredient percentage weight descriptor group includes one or more descriptors related to a percentage weight of the at least one type of non-monomer molecule in the pre-existing polymer, wherein the morphology percentage weight descriptor group includes one or more descriptors related to at least one of a percentage of hard segment in the pre-existing polymer, a percentage of soft segment in the pre-existing polymer, a percentage weight of the hard segment in the pre-existing polymer, a percentage weight of the soft segment in the pre-existing polymer, or any combination thereof, wherein the polymer parameter and stoichiometry descriptor group includes one or more descriptors related to at least one ratio between the at least one type of a group of atoms or bonds and at least one prepolymer of the pre-existing polymer, wherein the at least one descriptor of the plurality of pre-existing polymers from the at least three of the descriptor groups is associated with the at least one desired physical property for the plurality of pre-existing polymers, and wherein the one or more prediction scores include a prediction of one or more descriptors of the target polymer; and provide a formula of the target polymer, wherein the formula of the target polymer is based on the one or more prediction scores, and wherein the formula includes a plurality of raw materials of the target polymer and percentage weights of the plurality of raw materials of the target polymer.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure. As used in the specification and the claims, the singular form of “a,” “an,” and “the” include plural referents unless the context clearly dictates otherwise.

### BRIEF DESCRIPTION OF THE DRAWINGS

Additional advantages and details are explained in greater detail below with reference to the exemplary embodiments or aspects that are illustrated in the accompanying schematic figures, in which:

FIG. 1 is a diagram of a non-limiting embodiment or aspect of an environment in which systems, devices, products, apparatus, and/or methods, described herein, may be implemented;

FIG. 2 is a diagram of a non-limiting embodiment or aspect of components of one or more devices, systems, and/or networks of FIG. 1;

FIG. 3 is a flowchart of a non-limiting embodiment or aspect of a process for predicting a polymer;

FIG. 4 is a flowchart of a non-limiting embodiment or aspect of a process for predicting a polymer;

FIG. 5 is a flowchart of a non-limiting embodiment or aspect of a process for predicting a polymer;

FIG. 6 is a flowchart of a non-limiting embodiment or aspect of a process for predicting a polymer; and

FIG. 7 is a flowchart of a non-limiting embodiment or aspect of a process for predicting a polymer.

### DETAILED DESCRIPTION

In some non-limiting embodiments or aspects, a polymer, which for purposes of this disclosure also includes polymeric dispersions (e.g., a polyurethane dispersion (PUD), etc.), may be associated with one or more descriptors of the polymer (e.g., one or more quantitative descriptions of the polymer, etc.). For example, properties of a polymer may be known or modeled based on the one or more descriptors associated with the polymer.

However, each descriptor, on its own, may be a weak predictor of physical properties, including performance properties or characteristics, of polymers. For example, the one or more descriptors may not provide or indicate correlations between descriptors and physical properties of a polymer, whether a polymer is suitable for a particular application, similarities (or differences) between polymers with respect to descriptors and/or physical properties thereof, and/or the like. For example, a system may not include or recognize correlations between calculated descriptors (e.g., descriptors of the polymer calculated from descriptors of monomers or prepolymers of the polymer, such as, an atomic species count of the polymer, a monomer species count of the polymer, etc.) and measured descriptors (e.g., descriptors of measureable physical properties, including performance properties or characteristics, of the polymer, such as a tensile strength of the polymer, an adhesion of the polymer, an elongation of the polymer, a hardness of the polymer, a viscosity of the polymer when dispersed in water, etc.). As an example, existing information about a polymer may not be sufficient to determine relationships between calculated descriptors and physical properties of polymers, determine similar (or dissimilar) polymers based on descriptors and/or physical properties of the polymers, predict descriptors and/or raw materials of a polymer, and/or provide a useful representation of polymer properties across multiple polymers. Additionally, a polymer database may not be able to be generated that includes all desired polymers and all desired descriptors thereof if an individual provides manual designations for properties of the polymers (e.g., manual measurements of physical properties, including performance properties or characteristics, etc.) based on a lack of network and/or processing resources to generate the database, a lack of time to generate the database, and/or a lack of data to generate the database.

As disclosed herein, in some non-limiting embodiments or aspects, a polymer prediction system receives at least one descriptor of a target polymer, such as a polyurethane and/or PUD, from at least three descriptor groups described in more detail herein below; determines one or more prediction scores for the target polymer based on the at least one descriptor of the target polymer from the at least three of the descriptor groups and descriptor data including a plurality of descriptors of a plurality of pre-existing polymers, the plurality of descriptors being associated with one or more physical properties of the plurality of pre-existing polymers, and the one or more prediction scores including a prediction of the one or more physical properties for the target polymer; and provides physical property data associated with the target polymer, such as performance properties or characteristics of the target polymer (e.g., elongation, hardness, viscosity, adhesion, etc.), the physical property data being based on the one or more prediction scores.

As disclosed herein, in some non-limiting embodiments or aspects, a polymer prediction system receives physical property data including at least one desired physical property for a target polymer; determines one or more prediction scores for the target polymer based on the physical property data and at least one descriptor of a plurality of pre-existing polymers from at least three descriptor groups described in more detail herein below, the at least one descriptor of the plurality of pre-existing polymers from the at least three of the descriptor groups being associated with the at least one desired physical property for the plurality of pre-existing polymers, and the one or more prediction scores including a prediction of one or more descriptors of the target polymer; and provides a formula of the target polymer, the formula of the target polymer being based on the one or more prediction scores, the formula including a plurality of raw materials of the target polymer and percentage weights of the plurality of raw materials in the target polymer.

In this way, it has been found that the descriptors and descriptor groups discussed herein below, when descriptors from at least three different descriptor groups are used in combination, act collectively as a much stronger predictor of physical properties, including performance properties or characteristics, of polymers. For example, the polymer prediction system can determine relationships between calculated descriptors and physical properties of polymers, determine similar (or dissimilar) polymers based on descriptors of the polymers, predict physical properties of a polymer (e.g., without measuring a physical property of the polymer, etc.), predict raw materials and/or a formula of a polymer, and/or provide a more useful representation of polymer properties across multiple polymers. For example, a user interested in a target polymer (e.g., a new polymer for which limited information is known) can use the polymer prediction system to predict certain physical properties of the target polymer, such as certain performance properties or characteristics of the target polymer, based on existing data without having to physically create, test, and/or measure the target polymer. As an example, a user interested in a target polymer (e.g., a new polymer for which limited information is known) can use the polymer prediction system to predict raw materials of the target polymer based on existing data without having to physically create, test, and/or measure the target polymer. Additionally, a polymer database can be generated that includes all desired polymers and all desired descriptors and/or physical properties thereof. Accordingly, the inventors have found that a variety of descriptors from different areas, in particular, from areas as defined by those descriptor groups described in more detail herein below, when used in combination provide a better prediction of physical properties, including performance properties or characteristics, of polymers and, in particular, a much better prediction of physical properties, including performance properties or characteristics, of PUDs.

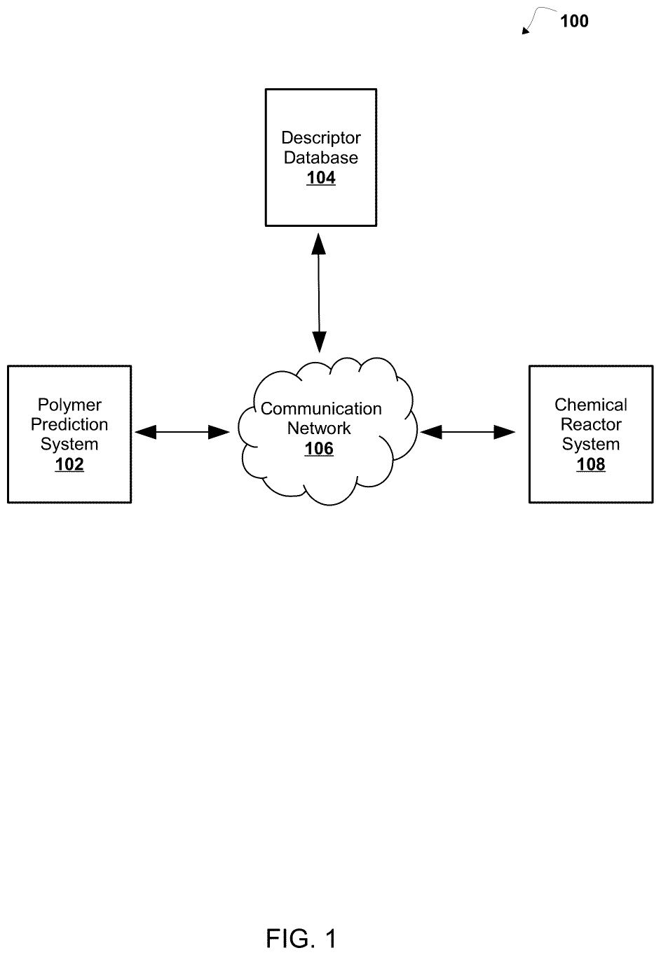

Referring now to FIG. 1, FIG. 1 is a diagram of an example environment 100 in which devices, systems, and/or methods, described herein, may be implemented. As shown in FIG. 1, environment 100 includes polymer prediction system 102, descriptor database 104, communication network 106, and/or chemical reactor system 108. Systems and/or devices of environment 100 may interconnect via wired connections, wireless connections, or a combination of wired and wireless connections.

In some non-limiting embodiments or aspects, polymer prediction system 102 includes one or more devices capable of receiving information from descriptor database 104 and/or chemical reactor system 108 and/or communicating information to descriptor database 104 and/or chemical reactor system 108 via communication network 106. For example, polymer prediction system 102 can include one or more computing systems including one or more processors (e.g., one or more servers, etc.). In some non-limiting embodiments or aspects, polymer prediction system 102 is capable of processing descriptor data including a plurality of descriptors of a plurality of pre-existing polymers and/or physical property data including one or more physical properties of the plurality of pre-existing polymers to generate or build one or more predictive models. In some non-limiting embodiments or aspects, polymer prediction system 102 is capable of processing a formula of a target polymer (e.g., descriptors of a target polymer and/or raw materials thereof, etc.) to generate one or more predictions (e.g., one or more prediction scores, etc.) of one or more physical properties, such as one or more performance characteristics, of the target polymer. For example, polymer prediction system 102 is capable of providing physical property data associated with the target polymer, and the physical property data is based on the one or more prediction scores. In some non-limiting embodiments or aspects, polymer prediction system 102 is capable of processing at least one desired physical property of a target polymer to generate one or more predictions (e.g., one or more prediction scores, etc.) of for one or more descriptors or raw materials of the target polymer. For example, polymer prediction system 102 is capable of providing a formula of the target polymer, the formula being based on the one or more prediction scores, and the formula including a plurality of raw materials of the target polymer and percentage weights of the plurality of raw materials of the target polymer. In some non-limiting embodiments or aspects, polymer prediction system 102 is implemented within chemical reactor system 108 or vice-versa.

In some non-limiting embodiments or aspects, descriptor database 104 includes one or more devices capable of receiving, storing, and/or providing descriptor data including a plurality of descriptors of a plurality of polymers and/or physical property data including a plurality of physical properties of the plurality of polymers. For example, descriptor database 104 can include one or more computing systems including one or more processors (e.g., one or more servers, etc.). In some non-limiting embodiments or aspects, descriptor database 104 includes one or more data structures for storing the descriptor data and/or the physical property data. In some non-limiting embodiments or aspects, polymer prediction system 102 includes descriptor database 104.

In some non-limiting embodiments or aspects, communication network 106 includes one or more wired and/or wireless networks. For example, communication network 106 can include a cellular network (e.g., a long-term evolution (LTE) network, a third generation (3G) network, a fourth generation (4G) network, a code division multiple access (CDMA) network, etc.), a public land mobile network (PLMN), a local area network (LAN), a wide area network (WAN), a metropolitan area network (MAN), a telephone network (e.g., the public switched telephone network (PSTN)), a private network, an ad hoc network, an intranet, the Internet, a fiber optic-based network, a cloud computing network, and/or the like, and/or a combination of these or other types of networks.

In some non-limiting embodiments or aspects, chemical reactor system 108 includes one or more devices capable of receiving information from polymer prediction system 102 and/or descriptor database 104 and/or communicating information to polymer prediction system 102 and/or descriptor database 104 via communication network 106. For example, chemical reactor system 108 can include one or more computing systems including one or more processors (e.g., one or more servers, etc.). In some non-limiting embodiments or aspects, chemical reactor system 108 includes a chemical reactor capable of producing a polymer from raw materials or ingredients in a chemical reaction. For example, chemical reactor system 108 may include one or more enclosed volumes (e.g., tanks, pipes, tubes, etc.) in which one or more chemical reactions can occur or be housed, one or more input ports via which raw materials or ingredients can be input to the one or more enclosed volumes, one or more output ports from which materials (e.g., an intermediate product or material of the one or more chemical reactions, a final product or material of the one more chemical reactions, a polymer produced by the one or more chemical reactions, etc.) can be output from the one or more enclosed volumes, one or more sensors capable of measuring one or more reaction parameters (e.g., a volume, a temperature, a pressure, a time, a concentration of a chemical species, raw material and/or ingredient, etc.) of the one or more chemical reactions and/or constituents thereof (e.g., a chemical reactor and components thereof, raw materials or ingredients, an input, an output, an intermediate product or material of the one or more chemical reactions, a final product or material of the one more chemical reactions, a polymer produced by the one or more chemical reactions, etc.), and/or one or more control devices (e.g., a heating device, a cooling device, a device to increase pressure (e.g., a pumping device, etc.), a device to decrease pressure (e.g., a valve, etc.), an agitator, etc.) capable of adjusting the one or more reaction parameters, an input of raw materials or ingredients via the one or more input ports, an output of products or materials from the one or more output ports, and/or the like for the one or more chemical reactions. Chemical reactor system 108 may include batch, continuous or hybrid chemical processes.

In some non-limiting embodiments or aspects, chemical reactor system 108 includes polymer prediction system 102 or vice-versa. For example, polymer prediction system 102 may be electrically and/or mechanically connected to the one or more control devices, the one or more sensors, the one or more input ports, and/or the one or more output ports of the chemical reactor of chemical reactor system 108, and polymer prediction system 102 may control the one or more control devices, the one or more sensors, the one or more input ports, and/or the one or more output ports to control chemical reactor system 108 to adjust the one or more reaction parameters, an input of the raw materials or ingredients via the one or more input ports, an output of the materials or products from the one or more output ports, and/or the like for the one or more chemical reactions based on the one or more reaction parameters measured during the one or more chemical reactions and/or one or more predictions of one or more physical properties of a polymer to be produced by the one or more chemical reactions.

The number and arrangement of devices and networks shown in FIG. 1 are provided as an example. There may be additional devices, systems, and/or networks, fewer devices, systems, and/or networks, different devices, systems, and/or networks, or differently arranged devices, systems, and/or networks than those shown in FIG. 1. Furthermore, two or more devices, systems, and/or networks shown in FIG. 1 may be implemented within a single device, system, and/or network, or a single device, system, and/or network shown in FIG. 1 may be implemented as multiple, distributed devices, systems, and/or networks. Additionally, or alternatively, a set of devices, systems, and/or networks (e.g., one or more devices, one or more systems, one or more networks, etc.) of environment 100 may perform one or more functions described as being performed by another set of devices, systems, and/or networks of environment 100.

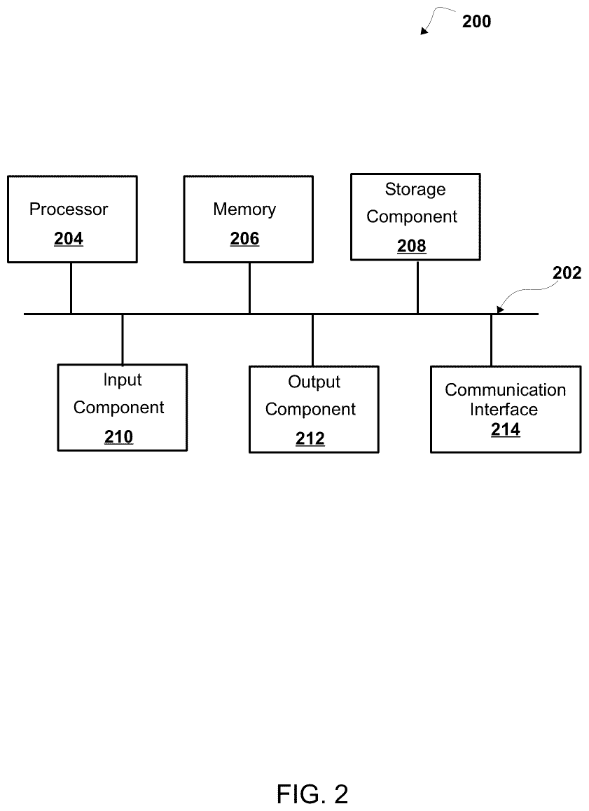

Referring now to FIG. 2, FIG. 2 is a diagram of example components of a device 200. Device 200 may correspond to one or more devices of polymer prediction system 102, one or more devices of descriptor database 104, one or more devices of communication network 106, and/or one or more devices of chemical reactor system 108. In some non-limiting embodiments or aspects, one or more devices of polymer prediction system 102, one or more devices of descriptor database 104, one or more devices of communication network 106, and/or one or more devices of chemical reactor system 108 can include at least one device 200 and/or at least one component of device 200. As shown in FIG. 2, device 200 may include a bus 202, a processor 204, memory 206, a storage component 208, an input component 210, an output component 212, and a communication interface 214.

Bus 202 may include a component that permits communication among the components of device 200. In some non-limiting embodiments or aspects, processor 204 may be implemented in hardware, firmware, or a combination of hardware and software. For example, processor 204 may include a processor (e.g., a central processing unit (CPU), a graphics processing unit (GPU), an accelerated processing unit (APU), etc.), a microprocessor, a digital signal processor (DSP), and/or any processing component (e.g., a field-programmable gate array (FPGA), an application-specific integrated circuit (ASIC), etc.) that can be programmed to perform a function. Memory 206 may include random access memory (RAM), read only memory (ROM), and/or another type of dynamic or static storage device (e.g., flash memory, magnetic memory, optical memory, etc.) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid-state disk, etc.), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touch screen display, a keyboard, a keypad, a mouse, a button, a switch, a microphone, etc.). Additionally, or alternatively, input component 210 may include a sensor for sensing information (e.g., a global positioning system (GPS) component, an accelerometer, a gyroscope, an actuator, etc.). Output component 212 may include a component that provides output information from device 200 (e.g., a display, a speaker, one or more light-emitting diodes (LEDs), etc.).

Communication interface 214 may include a transceiver-like component (e.g., a transceiver, a separate receiver and transmitter, etc.) that enables device 200 to communicate with other devices, such as via a wired connection, a wireless connection, or a combination of wired and wireless connections. Communication interface 214 may permit device 200 to receive information from another device and/or provide information to another device. For example, communication interface 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A memory device includes memory space located inside of a single physical storage device or memory space spread across multiple physical storage devices.

Software instructions may be read into memory 206 and/or storage component 208 from another computer-readable medium or from another device via communication interface 214. When executed, software instructions stored in memory 206 and/or storage component 208 may cause processor 204 to perform one or more processes described herein. Additionally, or alternatively, hardwired circuitry may be used in place of or in combination with software instructions to perform one or more processes described herein. Thus, embodiments or aspects described herein are not limited to any specific combination of hardware circuitry and software.

The number and arrangement of components shown in FIG. 2 are provided as an example. In some non-limiting embodiments or aspects, device 200 may include additional components, fewer components, different components, or differently arranged components than those shown in FIG. 2. Additionally, or alternatively, a set of components (e.g., one or more components) of device 200 may perform one or more functions described as being performed by another set of components of device 200.

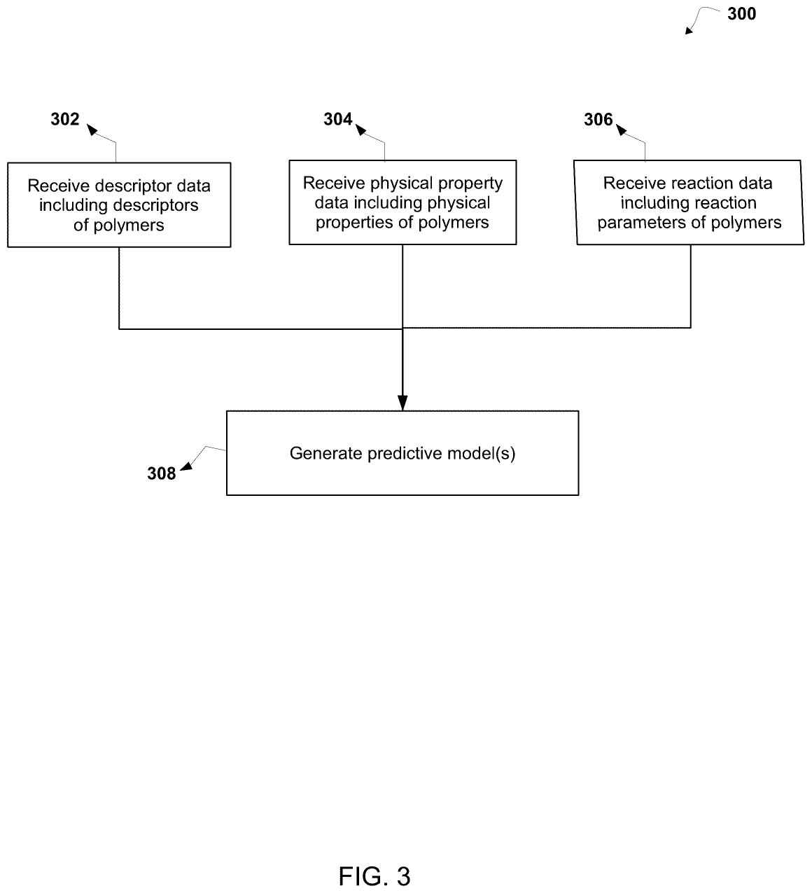

Referring now to FIG. 3, FIG. 3 is a flowchart of a non-limiting embodiment or aspect of a process 300 for predicting a polymer. In some non-limiting embodiments or aspects, one or more of the steps of process 300 may be performed (e.g., completely, partially, etc.) by polymer prediction system 102 (e.g., one or more devices of polymer prediction system 102). In some non-limiting embodiments or aspects, one or more of the steps of process 300 may be performed (e.g., completely, partially, etc.) by another device or a group of devices separate from or including polymer prediction system 102.

As shown in FIG. 3, at step 302, process 300 includes receiving descriptor data including a plurality of descriptors of a plurality of polymers. For example, polymer prediction system 102 receives descriptor data including a plurality of descriptors of a plurality of polymers. As an example, polymer prediction system 102 can receive or retrieve descriptor data including a plurality of descriptors of a plurality of polymers (e.g., a plurality of pre-existing polymers, etc.) from descriptor database 104.

In some non-limiting embodiments or aspects, a descriptor of a polymer includes a quantitative value that describes a calculable property of the polymer. For example, a polymer may be associated with (e.g., described by, defined by, etc.) one or more descriptors from one or more of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, a polymer parameter and stoichiometry descriptor group, and/or the like.

In some non-limiting embodiments or aspects, an atomic species count descriptor (e.g., a descriptor included in or selected from the atomic species count descriptor group, etc.) includes one or more descriptors related to a number of at least one type of atom per an amount (e.g., per kilogram, etc.) of a polymer. For example, an atomic species count descriptor may include at least one of the following descriptors of a polymer: a number of carbon atoms per an amount of the polymer, a number of hydrogen atoms per the amount of the polymer, a number of oxygen atoms per the amount of the polymer, a number of nitrogen atoms per the amount of the polymer, a number of sulfur atoms per the amount of the polymer, and/or the like.

In some non-limiting embodiments or aspects, a functional count descriptor (e.g., a descriptor included in or selected from the functional count descriptor group, etc.) includes one or more descriptors related to a number of at least one type of a group of atoms or bonds per an amount (e.g., per kilogram, etc.) of a polymer. For example, a functional count descriptor may include at least one of the following descriptors of a polymer: a number of carbonyl groups per an amount of the polymer, a number of isocyanate units per the amount of the polymer, a number of acid units per the amount of the polymer, a number of anhydride units per the amount of the polymer, a number of neutralizer units per the amount of the polymer, a number of carbonate links per the amount of the polymer, a number of ester links per the amount of the polymer, a number of urethane links per the amount of the polymer, a number of ether links per the amount of the polymer, a number of hydrogen acceptors per the amount of the polymer, a number of hydrogen donors per the amount of the polymer, a number of methyl (CH3) groups per the amount of the polymer, a number of methylene (CH2) groups per the amount of the polymer, a number of methane (CH) groups per the amount of the polymer, a number of carbon atoms without a hydrogen atom per the amount of the polymer, a number of acrylic acid groups per the amount of the polymer, a number of oils per the amount of the polymer, a number of two double bonds conjugated per the amount of the polymer, a number of three double bonds conjugated per the amount of the polymer, and/or the like.

In some non-limiting embodiments or aspects, a monomer species count descriptor (e.g., a descriptor included in or selected from the monomer species count descriptor group, etc.) includes one or more descriptors related to a number of at least one type of monomer per an amount (e.g., per kilogram, etc.) of a polymer. For example, a monomer species count descriptor may include at least one of the following descriptors of a polymer: a number of ethylene oxide groups per an amount of the polymer, a number of propylene oxide groups per the amount of the polymer, a number of hexanediol monomers per the amount of the polymer, a number of butanediol monomers per the amount of the polymer, a number of caprolactone monomers per the amount of the polymer, a number of moles of urea per the amount of the polymer, a number of moles of formaldehyde monomers per the amount of the polymer, a number of dicyclohexylmethane diisocyanate monomers (e.g., Desmodur® W monomers) per the amount of the polymer, a number of hexamethylene diisocyanate monomers (e.g., Desmodur® H monomers) per the amount of the polymer, a number of isophorone diisocyanate monomers (e.g., Desmodur® I monomers) per the amount of the polymer, a number of methylene diphenylmethane diisocyanate (MDI) monomers per the amount of the polymer, a number of tolylene diisocyanate (TDI) monomers per the amount of the polymer, a number of ethoxylated monol monomers (e.g., LB25 monomers) per the amount of the polymer, a number of propoxylated diol monomers (e.g., LP 112 monomers) per the amount of the polymer, a number of sulfonate monomers per the amount of the polymer, and/or the like.

In some non-limiting embodiments or aspects, an ingredient count descriptor (e.g., a descriptor included in or selected from the ingredient count descriptor group, etc.) includes one or more descriptors related to a number of at least one type of non-monomer molecule per an amount (e.g., per kilogram, etc.) of a polymer. For example, an ingredient count descriptor may include at least one of the following descriptors of a polymer: a number of polycarbonate molecules per the amount of the polymer, a number of polyester molecules per the amount of the polymer, a number of polyether molecules per the amount of the polymer, a number of diamine molecules (e.g., Dytek A molecules) per the amount of the polymer, a number of diacrylate ester of bisphenol A molecules (e.g., Ebecryl 600 molecules) per the amount of the polymer, a number of ethanol molecules per the amount of the polymer, a number of ethylene diamine molecules per the amount of the polymer, a number of formaldehyde molecules per the amount of the polymer, a number of gamma-butyrolactone molecules per the amount of the polymer, a number of hydrazine molecules per the amount of the polymer, a number of IPDA molecules per the amount of the polymer, a number of isopropanol molecules per the amount of the polymer, a number of 2-hydroxypropyl carbamate hydrazide molecules (e.g., KGD 1409 molecules) per the amount of the polymer, a number of L-Lysin molecules per the amount of the polymer, a number of polyester-modified acrylic oligomers (e.g., LaromerPE 44 F molecules, LVP WJD 478E molecules, etc.) per the amount of the polymer, a number of MDI based oligomers (e.g., MDI 1806 molecules) per the amount of the polymer, a number of Methoxypolyethyleneglycol molecules per the amount of the polymer, a number of Methylethylketoxime molecules per the amount of the polymer, a number of mono-n-butylamine molecules per the amount of the polymer, a number of monoethylene glycol molecules per the amount of the polymer, a number of neopentylglycol molecules per the amount of the polymer, a number of phthalic anhydride-hexane polyester molecules (e.g., P 200 H molecules) per the amount of the polymer, a number of hexanediol-neopentyl glycol-adipic acid polyester molecules (e.g., PE 170 HN A molecules) per the amount of the polymer, a number of butanediol-adipic acid polyester molecules (e.g., PE 225 B molecules) per the amount of the polymer, a number of butanediol-neopentyl glycol-adipic acid polyester molecules (e.g., PE 225 H molecules) per the amount of the polymer, a number of hexanediol-adipic acid polyester molecules (e.g., PE 84 H molecules) per the amount of the polymer, a number of butanediol-adipic acid polyester molecules (e.g., PE 90 B molecules) per the amount of the polymer, a number of polyethylene glycol monomethyl ether molecules per the amount of the polymer, a number of ethylene glycol-phthalic acid-adipic acid polyester hexanediol-isophthalic acid polyester molecules (e.g., PEI 200 H molecules) per the amount of the polymer, a number of ethylene glycol-phthalic acid-adipic acid polyester molecules (e.g., PEP 175 A molecules) per the amount of the polymer, and/or the like.

In some non-limiting embodiments or aspects, a bond type count descriptor (e.g., a descriptor included in or selected from the bond type count descriptor group, etc.) includes one or more descriptors related to a number of at least one type of bond per an amount (e.g., per kilogram, etc.) of a polymer. For example, a bond type count descriptor may include at least one of the following descriptors of a polymer: a number of linking bonds linking reactive groups per an amount of the polymer, a number of rotating bonds linking reactive groups per the amount of the polymer, a number of linking bonds and non-linking bonds per the amount of the polymer, and/or the like.

In some non-limiting embodiments or aspects, an atomic species percentage weight descriptor (e.g., a descriptor included in or selected from the atomic species percentage weight descriptor group, etc.) includes one or more descriptors related to a percentage weight of at least one type of atom in a polymer. For example, an atomic species percentage weight descriptor may include at least one of the following descriptors of a polymer: a percentage weight of carbon atoms in the polymer, a percentage weight of hydrogen atoms in the polymer, a percentage weight of oxygen atoms in the polymer, a percentage weight of nitrogen atoms in the polymer, a percentage weight of sulfur atoms in the polymer, and/or the like.

In some non-limiting embodiments or aspects, a functional percentage weight descriptor (e.g., a descriptor included in or selected from the functional percentage weight descriptor group, etc.) includes one or more descriptors related to a percentage weight of at least one type of a group of atoms or bonds in a polymer. For example, a functional percentage weight descriptor may include at least one of the following descriptors of a polymer: a percentage weight of carbonyl groups in the polymer, a percentage weight of isocyanate units in the polymer, a percentage weight of acid units in the polymer, a percentage weight of anhydride units in the polymer, a percentage weight of neutralizer units in the polymer, a percentage weight of carbonate links in the polymer, a percentage weight of ester links in the polymer, a percentage weight of urethane links in the polymer, a percentage weight of ether links in the polymer, a percentage weight of hydrogen acceptors in the polymer, a percentage weight of hydrogen donors in the polymer, a percentage weight of methyl (CH3) groups in the polymer, a percentage weight of methylene (CH2) groups in the polymer, a percentage weight of methane (CH) groups in the polymer, a percentage weight of carbon atoms without a hydrogen atom in the polymer, a percentage weight of acrylic acid groups in the polymer, a percentage weight of oils in the polymer, a percentage weight of two double bonds conjugated in the polymer, a percentage weight of three double bonds conjugated in the polymer, and/or the like.

In some non-limiting embodiments or aspects, a monomer percentage weight descriptor (e.g., a descriptor included in or selected from the monomer percentage weight descriptor group, etc.) includes one or more descriptors related to a percentage weight of at least one type of monomer in a polymer. For example, a monomer percentage weight descriptor may include at least one of the following descriptors of a polymer: a percentage weight of ethylene oxide groups in the polymer, a percentage weight of propylene oxide groups in the polymer, a percentage weight of hexanediol monomers in the polymer, a percentage weight of butanediol monomers in the polymer, a percentage weight of caprolactone monomers in the polymer, a percentage weight of moles of urea in the polymer, a percentage weight of moles of formaldehyde monomers in the polymer, a percentage weight of dicyclohexylmethane diisocyanate monomers (e.g., Desmodur® W monomers) in the polymer, a percentage weight of hexamethylene diisocyanate monomers (e.g., Desmodur® H monomers) in the polymer, a percentage weight of isophorone diisocyanate monomers (e.g., Desmodur® I monomers) in the polymer, a percentage weight of MDI monomers in the polymer, a percentage weight of TDI monomers in the polymer, a percentage weight of ethoxylated monol monomers (e.g., LB25 monomers) in the polymer, a percentage weight of propoxylated diol monomers (e.g., LP 112 monomers) in the polymer, a percentage weight of sulfonate monomers in the polymer, and/or the like.

In some non-limiting embodiments or aspects, an ingredient percentage weight descriptor (e.g., a descriptor included in or selected from the ingredient percentage weight descriptor group, etc.) includes one or more descriptors related to a percentage weight of at least one type of non-monomer molecule in a polymer. For example, an ingredient percentage weight descriptor may include at least one of the following descriptors of a polymer: a percentage weight of polycarbonate molecules in the polymer, a percentage weight of polyester molecules in the polymer, a percentage weight of polyether molecules in the polymer, a percentage weight of diamine molecules (e.g., Dytek A molecules) in the polymer, a percentage weight of diacrylate ester of bisphenol A molecules (e.g., Ebecryl 600 molecules) in the polymer, a percentage weight of ethanol molecules in the polymer, a percentage weight of ethylene diamine molecules in the polymer, a percentage weight of formaldehyde molecules in the polymer, a percentage weight of gamma-butyrolactone molecules in the polymer, a percentage weight of hydrazine molecules in the polymer, a percentage weight of IPDA molecules in the polymer, a percentage weight of isopropanol molecules in the polymer, a percentage weight of 2-hydroxypropyl carbamate hydrazide molecules (e.g., KGD 1409 molecules) in the polymer, a percentage weight of L-Lysin molecules in the polymer, a percentage weight of polyester-modified acrylic oligomers (e.g., LaromerPE 44 F molecules, LVP WJD 478E molecules, etc.) in the polymer, a percentage weight of MDI based oligomers (e.g., MDI 1806 molecules) in the polymer, a percentage weight of Methoxypolyethyleneglycol molecules in the polymer, a percentage weight of Methylethylketoxime molecules in the polymer, a percentage weight of mono-n-butylamine molecules in the polymer, a percentage weight of monoethylene glycol molecules in the polymer, a percentage weight of neopentylglycol molecules in the polymer, a percentage weight of phthalic anhydride-hexane polyester molecules (e.g., P 200 H molecules) in the polymer, a percentage weight of hexanediol-neopentyl glycol-adipic acid polyester molecules (e.g., PE 170 HN A molecules) in the polymer, a percentage weight of butanediol-adipic acid polyester molecules (e.g., PE 225 B molecules) in the polymer, a percentage weight of butanediol-neopentyl glycol-adipic acid polyester molecules (e.g., PE 225 H molecules) in the polymer, a percentage weight of hexanediol-adipic acid polyester molecules (e.g., PE 84 H molecules) in the polymer, a percentage weight of butanediol-adipic acid polyester molecules (e.g., PE 90 B molecules) in the polymer, a percentage weight of polyethylene glycol monomethyl ether molecules in the polymer, a percentage weight of ethylene glycol-phthalic acid-adipic acid polyester hexanediol-isophthalic acid polyester molecules (e.g., PEI 200 H molecules) in the polymer, a percentage weight of ethylene glycol-phthalic acid-adipic acid polyester molecules (e.g., PEP 175 A molecules) in the polymer, and/or the like.

In some non-limiting embodiments or aspects, a morphology percentage weight descriptor (e.g., a descriptor included in or selected from the morphology percentage weight descriptor group, etc.) includes one or more descriptors related to at least one of a percentage of hard segment in the polymer, a percentage of soft segment in the polymer, a percentage weight of hard segment in the polymer, a percentage weight of soft segment in the polymer, or any combination thereof. As an example, the morphology percentage weight descriptor group may include the following descriptors of a polymer: a percentage of hard segment in the polymer, a percentage of soft segment in the polymer, a percentage weight of hard segment in the polymer, a percentage weight of soft segment in the polymer, and/or the like.

In some non-limiting embodiments or aspects, a polymer parameter and stoichiometry descriptor (e.g., a descriptor included in or selected from the polymer parameter and stoichiometry descriptor group, etc.) includes one or more descriptors related to at least one ratio between at least one type of a group of atoms or bonds and at least one prepolymer of a polymer. For example, a polymer parameter and stoichiometry descriptor may include at least one of the following descriptors of a polymer: a prepolymer number average molecular weight of the polymer, a prepolymer weight average molecular weight of the polymer, a prepolymer polydispersity of the polymer, an amount of urea per an amount of the polymer, a ratio of an amount of urea from amine to an amount of urethane in the polymer, a ratio of an amount of urea from amine to an amount of urethane and urea in the polymer, an amount of urea and urethane per an amount of the polymer, a ratio of NCO equivalents to OH equivalents in the polymer, a ratio of NCO equivalent to NH equivalents in the polymer, and/or the like.

However, it should be noted that one or more of the descriptor groups discussed above may not include each of the descriptors described as being associated with that descriptor group. Rather, the identified descriptors are representative descriptors of the type that may be included in each of the descriptor groups. The particular descriptors that make up a particular descriptor group for a particular polymer may depend, for example, on the details or properties of the polymer (e.g., which atoms are contained in the polymer, etc.) as well as the information that has been provided or entered into descriptor database 104 for that polymer. Similarly, certain polymers may have descriptors from less than all of the above-described descriptor groups associated therewith. In other words, some polymers may not have associated therewith any descriptors in certain of the described descriptor groups.

As further shown in FIG. 3, at step 304, process 300 includes receiving physical property data including one or more physical properties of the plurality of polymers. For example, polymer prediction system 102 receives physical property data including one or more physical properties of the plurality of polymers. As an example, polymer prediction system can receive or retrieve physical property data including one or more physical properties of the plurality of polymers from descriptor database 104.

In some non-limiting embodiments or aspects, a physical property, including a performance property or characteristic, of a polymer includes a quantitative value that describes a measurable physical property of the polymer, such as a tensile strength of the polymer, an adhesion of the polymer, an elongation of polymer, a hardness of the polymer a viscosity of the polymer in water, and/or the like. For example, a physical property may include at least one of the following physical properties of a polymer: a cost of the polymer (e.g., a manufacturing cost associated with producing the polymer, a cost of the polymer for a customer, etc.), a mean percentage of solids of the polymer, a solids method of the polymer, a minimum flow per time (e.g., per second, etc.) of the polymer, a maximum flow per time (e.g., per second, etc.) of the polymer, a flow cup of the polymer, a minimum viscosity (e.g., a minimum mPa·s, etc.) of the polymer, a maximum viscosity (e.g., a maximum mPa·s, etc.) of the polymer, a viscosity method of the polymer, a pH of the polymer, a number of acids from RS of the polymer, a number of OH #, a particle size (e.g., in nm, etc.) of the polymer, a modulus at 100% (e.g., in psi, etc.) of the polymer, an ultimate strength (e.g., in psi, etc.) of the polymer, an elongation percentage of the polymer, a softening point (e.g., in Celsius, etc.) of the polymer, an adhesion PVC/PVC (e.g., in pli, etc.) of the polymer, an adhesion PVC/Wood (e.g., in pli, etc.) of the polymer, a heat activation (e.g. in Celsius, etc.) of the polymer, a Tg (e.g., in Celsius, etc.) of the polymer, a microhardness (e.g., in N/mm2, etc.) of the polymer, a pendulum hardness at 1 day of the polymer, a pendulum hardness at 7 days of the polymer, a chemical residue of H2SO4 of the polymer, a chemical residue of NaOH 10% of the polymer, a chemical residue of ethanol of the polymer, a chemical residue of water of the polymer, a chemical residue of Xylene of the polymer, a hydrolytic stability at 1 week of the polymer, a hydrolytic stability at 2 weeks of the polymer, a dry time TF (e.g., in minutes, etc.) of the polymer, a dry time HD (e.g., in minutes, etc.) of the polymer, a Mn (e.g., in g/mol, etc.) of the polymer, a Mw (e.g., in g/mol of the polymer, etc.), a direct impact (e.g., in·lb, etc.) of the polymer, an inverse impact (e.g., in·lb, etc.) of the polymer, a Taber abrasion (e.g., in mg, etc.) of the polymer, a Taber comment of the polymer, a gloss 1 day 20° [GU] of the polymer, a gloss 3 day 20° [GU] of the polymer, a gloss 7 day 20° [GU] of the polymer, a cross hatch test of the polymer, and/or the like.

In some non-limiting embodiments or aspects, performance properties or characteristics of a polymer may indicate user measured field performance properties or characteristics of the polymer (and/or predicted values of the polymer for user measured field performance properties or characteristics of the polymer). For example, physical properties of a polymer, such as a solids method used to measure a percentage of solids of the polymer, a flow cup type used to measure a flow rate of the polymer, an adhesion of PVC to PVC provided by the polymer, an adhesion of PVC to wood provided by the polymer, a heat activation temperature of the polymer, a glass transition temperature of the polymer, a microhardness of the polymer, a pendulum hardness measured at 1 day after application of the polymer, a pendulum hardness measured at 7 days after application of the polymer, a chemical residue of H2SO4 of the polymer, a chemical residue of NaOH 10% of the polymer, a chemical residue of ethanol of the polymer, a chemical residue of water of the polymer, a chemical residue of Xylene of the polymer, a hydrolytic stability at 1 week after application of the polymer, a hydrolytic stability at 2 weeks after application of the polymer, a dry time TF of the polymer, a dry time HD of the polymer, a direct impact strength of the one or more polymers, an inverse impact strength of the one or more polymers, a Taber abrasion of the one or more polymers, a Taber comment of the one or more polymers, a gloss level at 1 day after application of the polymer, a gloss level at 3 days after application of the polymer, a gloss level at 7 days after application of the polymer, results of a cross hatch test of the polymer, and/or the like, may be user measured field performance properties or characteristics of the polymer (and/or predicted values of the polymer for the user measured field performance properties or characteristics of the polymer).

As further shown in FIG. 3, at step 306, process 300 includes receiving reaction data including one or more reaction parameters. For example, polymer prediction system 102 receives reaction data including one or more reaction parameters. As an example, polymer prediction system 102 can receive or retrieve reaction data including a plurality of reaction parameters measured during a plurality of chemical reactions (e.g., a plurality of previously performed chemical reactions, etc.) in chemical reactor system 108 (e.g., from a database of chemical reactor system 108, from the one or more sensors of chemical reactor system 108, etc.) for producing a plurality of polymers.

In some non-limiting embodiments or aspects, a reaction parameter includes a quantitative value and/or a qualitative value of a chemical reaction and/or constituents thereof (e.g., a chemical reactor (and/or components thereof), raw materials or ingredients, a material input to the chemical reaction, an intermediate product or material of the chemical reaction, a final product or material of the chemical reaction, a polymer produced by the chemical reaction, etc.) measured or determined during the chemical reaction. For example, a reaction parameter may include any value capable of being sensed, monitored, or determined (e.g., by the one or more sensors of chemical reactor system 108, etc.) during a chemical reaction, such as at least one of a residence time of a material of the chemical reaction, a volume of a material of the chemical reaction, a volume of an enclosed volume of the chemical reactor for the chemical reaction, a temperature of a material of the chemical reaction, a temperature of a component (e.g., the enclosed volume, a heater, etc.) of the chemical reactor for the chemical reaction, a pressure of material of the chemical reaction, a pressure within an enclosed volume of the chemical reactor for the chemical reaction, concentrations and/or amounts of products, materials, and/or ingredients in the chemical reaction, and/or the like, including but not limited to order of addition of the ingredients, rate of addition of the ingredients, speed of mixing, ramping of temperature and rate of pressure change.

In some non-limiting embodiments or aspects, reaction data is associated with a material, a product, and/or a polymer produced during and/or by a chemical reaction (and/or a formula including raw materials or ingredients used to produce the material, product, and/or polymer in the chemical reaction). For example, a reaction parameter may be associated with one or more physical properties, including performance characteristics, of a material, a product, and/or a polymer produced during and/or by a chemical reaction in which (e.g., during, occurring at the same time as, etc.) the reaction parameter is measured or determined (and/or one or more physical properties of one or more materials measured in and/or during the chemical reaction for producing the polymer). As an example, a reaction parameter may be associated with a viscosity of the material, product, and/or produced polymer, a turbidity of the material, product, and/or produced polymer, a color of the material, product, and/or produced polymer, or any other physical property or performance character of the material, product, and/or produced polymer.

As further shown in FIG. 3, at step 308, process 300 includes generating one or more predictive models. For example, polymer prediction system 102 generates one or more predictive models. As an example, polymer prediction system 102 generates one or more predictive models based on the descriptor data including the plurality of descriptors of the plurality of polymers (e.g., a plurality of pre-existing polymers, etc.), the physical property data including one or more physical properties of the plurality of polymers, and/or the reaction data including the one or more reaction parameters of the plurality of polymers. The types of data shown in steps 302, 304, and 306 in FIG. 3 as input for generating one or more predictive models are provided as an example. In some non-limiting embodiments or aspects, process 300 may include additional types of data, fewer types of data, or different types of data, than those shown in FIG. 3 as input for generating one or more predictive models.

In some non-limiting embodiments or aspects, polymer prediction system 102 generates the one or more predictive models for determining one or more prediction scores based on a machine learning technique (e.g., a pattern recognition technique, a data mining technique, a heuristic technique, a supervised learning technique, an unsupervised learning technique, a random forest technique, etc.). For example, polymer prediction system 102 generates the one or more predictive models (e.g., an estimator, a classifier, a prediction model, etc.) based on a machine learning algorithm (e.g., a decision tree algorithm, a gradient boosted decision tree algorithm, a neural network algorithm, a convolutional neural network algorithm, a random forest algorithm, etc.). In such an example, polymer prediction system 102 generates the one or more prediction scores using the one or more predictive models.

In some non-limiting embodiments or aspects, the one or more predictive models are designed to receive, as an input, descriptors of raw materials and/or a formula of the raw materials in a target polymer, and provide, as an output, a prediction (e.g., a probability, a binary output, a yes-no output, a score, a prediction score, etc.) as to one or more physical properties (e.g., performance characteristics) of the target polymer (e.g., as to whether the target polymer includes a physical property or performance characteristic, as to a quantitative value of the physical property or performance characteristic, etc.). For example, polymer prediction system 102 generates one or predictive models for predicting one or more physical properties, including one or more performance characteristics, of a target polymer. As an example, polymer prediction system 102 can generate the one or more predictive models to determine one or more prediction scores that include a prediction of whether the target polymer includes the one or more physical properties and/or a prediction of one or more quantitative values of the one or more physical properties of the target polymer. In such an example, polymer prediction system 102 can generate the one or more predictive models to be configured to predict one or more currently unknown measurable physical properties of a target polymer based on input to the one or more predictive models that includes descriptors of raw materials and/or a formula of the raw materials in the target polymer.

In some non-limiting embodiments or aspects, the one or more predictive models are designed to receive, as an input, one or more desired physical properties and/or optimization values of the target polymer, and provide, as an output, a prediction (e.g., a probability, a binary output, a yes-no output, a score, a prediction score, etc.) as to whether the target polymer includes one or more raw materials and/or a formula for the raw materials in the target polymer. For example, polymer prediction system 102 generates one or more predictive models for predicting one or more raw materials and/or a formula of the raw materials for a target polymer. As an example, polymer prediction system 102 can generate the one or more predictive models to determine one or more prediction scores that include a prediction of whether the target polymer includes one or more raw materials and/or a formula for the raw materials in the target polymer. In such an example, polymer prediction system 102 can generate the one or more predictive models to be configured to predict one or more currently unknown raw materials and/or a formula of the currently unknown raw materials in the target polymer based on input to the one or more predictive models that includes one or more desired physical properties of the target polymer and/or one or more optimization values of the desired physical properties of the target polymer.

In some non-limiting embodiments or aspects, the one or more predictive models are designed to receive, as an input, a formula of raw materials or ingredients for a polymer to be produced by a chemical reaction and one or more reaction parameters measured by one or more sensors of chemical reactor system 108 during the chemical reaction for producing the polymer, and provide, as an output, a prediction (e.g., a probability, a binary output, a yes-no output, a score, a prediction score, a target value, etc.) as to one or more physical properties (e.g., performance characteristics) of the polymer being produced by the chemical reaction (e.g., as to whether the formula and measured reaction parameters can produce a polymer that includes a desired physical property or performance characteristic, as to a quantitative value of the physical property or performance characteristic of a polymer to be produced by the formula and measured reaction parameters, etc.). For example, polymer prediction system 102 generates one or more predictive models for predicting one or more physical properties, including one or more performance characteristics, of the polymer to be produced by the chemical reaction based on the formula and the measured reaction parameters. As an example, polymer prediction system 102 generates one or more predictive models for adjusting reaction parameters (e.g., for controlling the one or more control devices of chemical reactor system 108 to adjust reaction parameters, etc.) of a chemical reaction in chemical reactor system 108 to produce a polymer having one or more desired or targeted physical properties (e.g., performance characteristics, etc.) associated with the formula for the polymer. As an example, polymer prediction system 102 can generate the one or more predictive models to determine or predict a drift in one or more physical properties of a polymer being produced during a chemical reaction in chemical reactor system 108, and control chemical reactor system 108 to adjust the one or more reaction parameters during the chemical reaction to correct for or inhibit the drift in the one or more physical properties of the polymer being produced in the chemical reaction.

In some non-limiting embodiments or aspects, polymer prediction system 102 stores the one or more predictive models (e.g., stores the model(s) for later use). In some non-limiting embodiments or aspects, polymer prediction system 102 stores the one or more predictive models in a data structure (e.g., a database, a linked list, a tree, etc.). In some non-limiting embodiments or aspects, the data structure is located within polymer prediction system 102 or external (e.g., remote from) polymer prediction system 102.

In some non-limiting embodiments or aspects, polymer prediction system 102 processes the descriptor data and/or the physical property data to obtain training data for the one or more models. For example, polymer prediction system 102 processes the descriptor data and/or the physical property data to change the descriptor data and/or the physical property data into a format that is analyzed (e.g., by polymer prediction system 102) to generate the one or more models. The descriptor data and/or the physical property data that is changed is referred to as training data. In some implementations, polymer prediction system 102 processes the descriptor data and/or the physical property data to obtain the training data based on receiving the descriptor data and/or the physical property data. Additionally, or alternatively, polymer prediction system 102 processes the descriptor data and/or the physical property data to obtain the training data based on polymer prediction system 102 receiving an indication that polymer prediction system 102 is to process the descriptor data and/or the physical property data from a user of polymer prediction system 102, such as when polymer prediction system 102 receives an indication to create a model for a plurality of polymers.

In some non-limiting embodiments or aspects, polymer prediction system 102 processes the descriptor data and/or the physical property data by determining one or more variables based on the descriptor data and/or the physical property data. In some non-limiting embodiments or aspects, a variable includes a metric, associated with a descriptor and/or a physical property of the polymer, which may be derived based on the descriptor data and/or the physical property data. The variable is analyzed to generate a model. For example, the variable includes a variable associated with a descriptor of a polymer and/or a physical property of a polymer.

In some non-limiting embodiments or aspects, polymer prediction system 102 analyzes the training data to generate a model (e.g., the one or more prediction models). For example, polymer prediction system 102 uses machine learning techniques to analyze the training data to generate the model. In some implementations, generating the model (e.g., based on training data obtained from descriptor data, based on training data obtained from pre-existing descriptor data) is referred to as training the model. The machine learning techniques include, for example, supervised and/or unsupervised techniques, such as decision trees (e.g., gradient boosted decision trees), logistic regressions, artificial neural networks (e.g., convolutional neural networks), Bayesian statistics, learning automata, Hidden Markov Modeling, linear classifiers, quadratic classifiers, association rule learning, random forests, and/or the like. In some non-limiting embodiments or aspects, the model includes a prediction model that is specific to a particular plurality of polymers, one or more particular physical properties, one or more particular descriptors, a particular application of a plurality of polymers, and/or the like. Additionally, or alternatively, the model is specific to a particular type of polymer (e.g., a polymer dispersion, a PUD, a type of PUD, etc.). In some implementations, polymer prediction system 102 generates one or more prediction models for one or more types of polymers, a particular group of types of polymers, and/or the like.

Additionally, or alternatively, when analyzing the training data, polymer prediction system 102 identifies one or more variables (e.g., one or more independent variables) as predictor variables that are used to make a prediction (e.g., when analyzing the training data). In some implementations, values of the predictor variables are inputs to the model. For example, polymer prediction system 102 identifies a subset (e.g., a proper subset) of variables as predictor variables that are used to accurately predict one or more physical properties of a polymer. As an example, polymer prediction system 102 identifies a subset (e.g., a proper subset) of variables as predictor variables that are used to accurately predict one or more raw materials and/or a formula of the raw materials of a polymer. In some implementations, the predictor variables include one or more of the variables, as discussed above, that have a significant impact (e.g., an impact satisfying a threshold) on a probability that the polymer includes one or more physical properties and/or on a probability that the polymer includes one or more raw materials and/or a formula of the raw materials.

In some non-limiting embodiments or aspects, polymer prediction system 102 validates the one or more models. For example, polymer prediction system 102 validates the one or more models after polymer prediction system 102 generates the one or more models. In some implementations, polymer prediction system 102 validates the one or more models based on a portion of the training data to be used for validation. For example, polymer prediction system 102 may partition the training data into a first portion and a second portion, where the first portion is used to generate the one or more models, as described above. In this example, the second portion of the training data (e.g., the validation data) is used to validate the one or more models. In some non-limiting embodiments or aspects, the first portion of the training data is different from the second portion of the training data.

In some non-limiting embodiments or aspects, polymer prediction system 102 validates the model by providing validation data including descriptor data that includes a plurality of descriptors of a plurality of polymers as input to the model, and determining, based on an output of the prediction model, whether the prediction model correctly, or incorrectly, predicted the one or more physical properties of the plurality of polymers. In some non-limiting embodiments or aspects, polymer prediction system 102 validates the model by providing validation data including physical property data that includes one or more physical properties of a plurality of polymers as input to the model, and determining, based on an output of the prediction model, whether the prediction model correctly, or incorrectly, predicted the raw materials and/or the formulas of the raw materials of the plurality of polymers.

In some implementations, polymer prediction system 102 validates the model based on a validation threshold (e.g., a threshold value of the validation data). For example, polymer prediction system 102 is configured to validate the model when a polymer (e.g., one or more physical properties of a polymer, raw materials and/or a formula of the raw materials of a polymer, etc.) is correctly predicted by the model (e.g., when the prediction model correctly predicts 50% of the validation data, when the prediction model correctly predicts 70% of the validation data, etc.). In some non-limiting embodiments or aspects, if polymer prediction system 102 does not validate a model (e.g., when a percentage of validation data does not satisfy the validation threshold), polymer prediction system 102 generates additional prediction models.

In some non-limiting embodiments or aspects, if the one or more models have been validated, polymer prediction system 102 further trains the one or more models and/or creates new models based on receiving new training data. In some non-limiting embodiments or aspects, the new training data includes descriptor data and/or physical property data associated with a plurality of polymers that is different from a previous plurality of polymers previously used to train the one or more models.

In a non-limiting embodiment or aspect of an implementation related to step 308 in process 300 shown in FIG. 3, specifically a non-limiting embodiment or aspect of how polymer prediction system 102 generates a predictive model in step 308 of process 300 for determining a prediction of a tensile strength (e.g., a logarithm of tensile strength in psi at 100% elongation) for a yet un-tested PUD (e.g., a target PUD, etc.), polymer prediction system 102 may use data (e.g., descriptor data, physical property data, etc.) on known or tested PUDs (e.g. 82 PUDs for which raw materials, formulas of raw materials, and tensile strength is known, etc.), calculate the independent variables (e.g., quantitative descriptors that describe the PUDs) based on descriptors of raw materials or components of the PUDs and the stoichastic relationship of the reactive groups in the formulas and for which the desired physical property of tensile strength has been measured. For example, polymer prediction system 102 may split the data on the known or tested 82 PUDs into two groups of data, one group of data for training the predictive model (e.g., data on a selected 67 PUDs), and the other group of data for testing and validating the predictive model (e.g., data on a remaining 15 PUDs). In another implementation, all of the data on the PUDs is used for training, and none of the data on the PUDs is used for validation. Polymer prediction system 102 may use the training set (e.g., the data on the selected 67 of the set of 82 PUDs) to train various machine learning algorithms (e.g., a Cubist algorithm, a multivariate adaptive regression splines (MARS) algorithm, a Random Forest algorithm, a partial least squares (PLS) algorithm, a support vector machine (SVM) algorithm, a classification and regression tree (CART) algorithm, etc.) using the dependent variable of tensile strength at 100% elongation and the calculated independent variables of the selected training PUDs. In some non-limiting embodiments or aspects, predictions from multiple machine learning algorithms may be combined to build an ensemble of algorithms or predictive models to increase predictive power.

In such an implementation, polymer prediction system 102 selects a machine learning algorithm or model from a plurality of machine learning algorithms or models based on an ability of the algorithm or model to predict the training data. For example, polymer prediction system 102 may compare an accuracy of the various models in predicting the training data, for example, the log-tensile strength of the selected 67 PUDs, by plotting the predicted log-tensile strength versus measured log-tensile strength, calculating the sum of squares of the prediction residuals, and selecting one or more of the various models based on the sum of squares of the prediction residuals (e.g., by selecting one or more models with a mean square of the residuals that satisfy one or more threshold values, by selecting at least one model with a lowest mean square of the residuals, etc.). In such an implementation, polymer prediction system 102 may further validate and test the out-of-sample predictive power of the algorithms or models by inputting the descriptor data (e.g., the quantitative descriptors) of the unselected remaining 15 PUDs of the set of 82 PUDS into the trained prediction models and comparing the predicted log-tensile strengths of the remaining 15 PUDs to measured values of the log-tensile strengths of the remaining PUDs. For example, polymer prediction system 102 may use the selected models to predict the log-tensile strength of the remaining 15 out-of-sample test PUDs using the calculated polymeric descriptors of the remaining 15 out-of-sample test PUDs as input to the models and compare the predicted values of the tensile strengths of the 15 PUDs with measured values of the tensile strengths of the 15 PUDS to assess an accuracy of the models for the 15 out-of-sample PUDs. In such an example, polymer prediction system 102 may build an ensemble model by averaging predictions of selected models, comparing plots with sum of squares of the residuals to individual models, and selecting an ensemble model or an individual model based on the comparison.

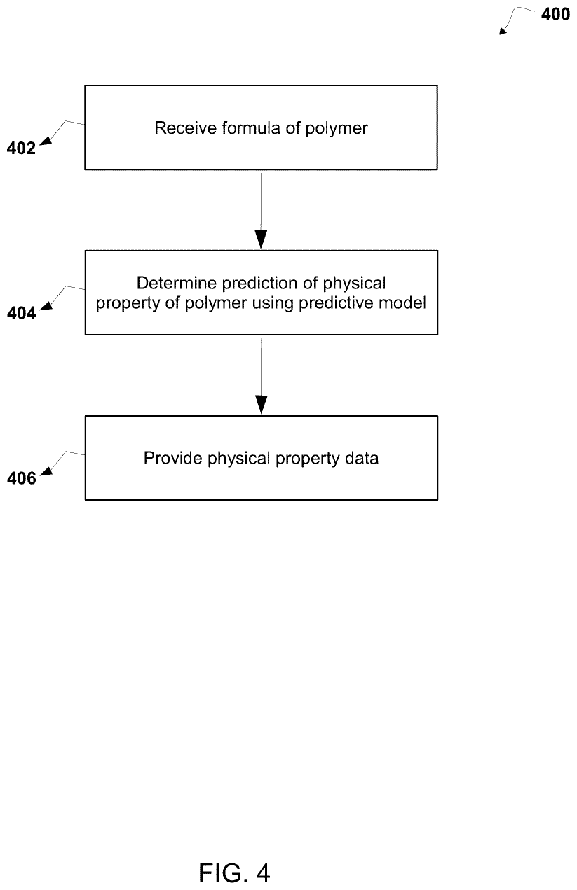

Referring now to FIG. 4, FIG. 4 is a flowchart of a non-limiting embodiment or aspect of a process 400 for predicting a polymer. In some non-limiting embodiments or aspects, one or more of the steps of process 400 may be performed (e.g., completely, partially, etc.) by polymer prediction system 102 (e.g., one or more devices of polymer prediction system 102). In some non-limiting embodiments or aspects, one or more of the steps of process 400 may be performed (e.g., completely, partially, etc.) by another device or a group of devices separate from or including polymer prediction system 102.

As shown in FIG. 4, at step 402, process 400 includes receiving a formula of a polymer. For example, polymer prediction system 102 receives a formula of a polymer. As an example, polymer prediction system 102 receives a formula of a target polymer including descriptors of raw materials in the polymer and percentage weights of the raw materials in the polymer from user input and/or descriptor database 104.

In some non-limiting embodiments or aspects, polymer prediction system 102 receives at least one descriptor of a target polymer from at least three of the descriptor groups defined herein above. For example, polymer prediction system 102 receives at least one descriptor of a target polymer from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group. It has been discovered that there is a correlation between descriptors from the above-noted descriptor groups and the ability to predict physical properties, including performance properties or characteristics, of a target polymer through the process described herein. For example, each descriptor, on its own, may be a weak predictor of physical properties, including performance properties or characteristics, of polymers; however, it has been found that the descriptors and descriptor groups discussed herein, when descriptors from at least three different descriptor groups are used in combination, act collectively as a much stronger predictor of physical properties, including performance properties or characteristics, of polymers.

In some non-limiting embodiments or aspects, polymer prediction system 102 receives descriptors of a target polymer based upon user input about the general structure or composition of the target polymer and information contained in descriptor database 104. For example, polymer prediction system 102 may receive descriptors of a target polymer from a user through a request for a prediction of one or more currently unknown physical properties or performance characteristics of the target polymer. As an example, polymer prediction system 102 may retrieve (e.g., in response to a user request) descriptors of a target polymer from descriptor database 104 for which one or more physical properties or performance characteristics are currently unknown.

By way of example, descriptor database 104 can include information about raw materials that can be blended together to create a target polymer. Common raw materials include, but are not limited to, water, polyols, and additives such as solvents. Descriptor database 104 can include, for each such raw material, a variety of descriptors, such as a concentration of molecular and polymer entities in a polymer (e.g., with the concentration expressed as weight or count per unit of weight or volume, such as percent by weight (or grams per kg), grams per liter, moles per kg, or moles per liter, and/or the like, the molecular formula, molecular weight, molar mass, number of each atom, etc.) and a stoichiometric ratio of reactive groups in a polymerization process. As an example, molecular and polymer entities in a polymer may include entities such as one or more of the following: atoms (e.g., moles of carbon atoms in a liter of a polymer, a weight of all hydrogen atoms in a kilogram of a polymer, etc.); bonds, which may be quantified according to the following three sub-categories and any combinations of the three subcategories: atom bounded and multiplicity of bonds (e.g., a number of carbon-carbon single bonds, carbon-carbon double bonds, carbon-carbon triple bonds, carbon-oxygen single bond, carbon-oxygen double bond, etc.), location bond in reference to the polymer backbone (e.g., a number of bonds in the polymer backbone per liter, a number of bonds in the danglers per kilogram, etc.), rotatable (e.g., a number of bonds that can rotate, a number of bonds that cannot rotate in a kilogram, etc.), combinations of any two or more of the three sub-categories (e.g., a number of bonds in the backbone that can rotate per kilogram, etc.); linking groups (e.g., a number of urethane links in a kilogram, a number of urethane links in a kilogram, a number of ether links in a liter, a weight of polyester links, such as the weight of the atoms in C(═O)O] in a kilogram of polymer, etc.); alkyl structures and rings (e.g., a number of cyclohexyl groups per kilogram, a number of n-hexamethylenes per kilogram, etc.); monomer units (e.g., a number of ethylene oxides molecules (used to make any polyether present in the polymer) per kg of polymer, a number of MDI monomers per kg of polymer, a number of adipic acid (used to make the polyesters that are an ingredient in the polymerization) per kg of final polymer, etc.); oligomeric ingredients (e.g., a percent weight of polymer that is a polyester, a number of polyester polyol per kg of polymer, etc.); specific ingredients (e.g., a percent weight Desmophen C-2200 in the polymer, etc.); morphological/phenomenological effect of the ingredient (e.g., a percent weight of the ingredients that segment into the polyurethane hard segment, a percent weight of the ingredients that impart dispensability in water to the polymer, etc.); and/or the like. As an example, stoichiometric ratio of reactive groups in a polymerization process may include indexes such as an overall index (e.g., a number of isocyanate groups divided by a number of isocyanate reactive groups in a final step when making the polymer, etc.); a specific index (e.g., a number of isocyanate groups divided by a number of alcohol reactive groups, etc.); and/or the like. Descriptor database 104 can be periodically updated to add materials or to include new properties of existing materials.

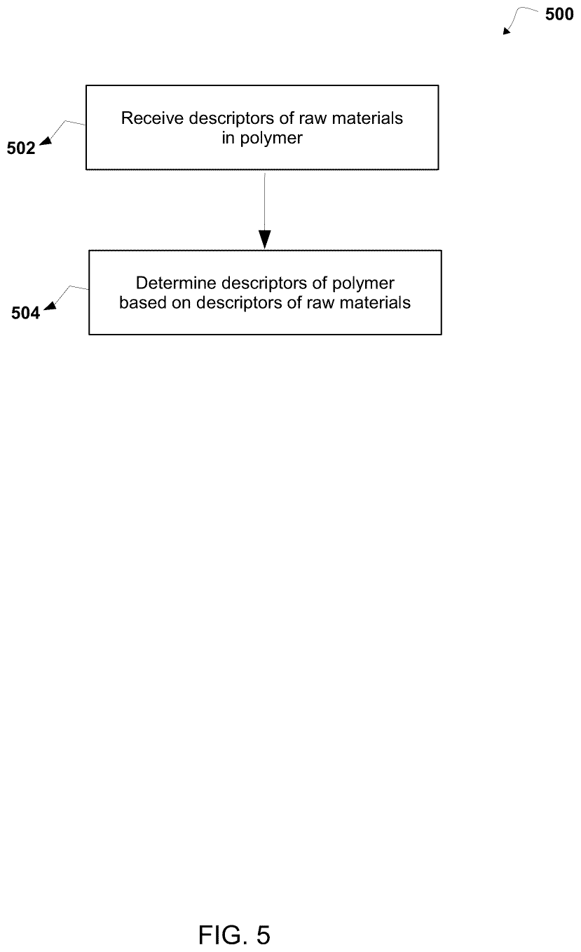

In some non-limiting embodiments or aspects, and referring also to FIG. 5, which is a flowchart of a non-limiting embodiment or aspect of a process 500 for predicting a polymer, at step 502 in process 500, polymer prediction system 102, having been provided with the identity and possibly amount of each of a plurality of raw materials in a target polymer from, for example, a user, receives a plurality of descriptors of the raw materials from descriptor database 104, and, at step 504 in process 500, polymer prediction system 102 determines one or more descriptors of the target polymer (such as descriptors falling within the categories described above) based on the plurality of descriptors of the plurality of raw materials.

For example, polymer prediction system 102 calculates descriptors for a polymer from descriptors of starting raw materials or ingredients of the polymer. This can be done, for example, by recursively calculating descriptors of a polymer by decomposing the polymer into each of the monomers used to create the polymer. As an example, the starting raw materials can be monomers (e.g., hexamethylene diisocyanate monomers, adipic acid, ethylene oxide, etc.), oligomers (e.g., hexanediol-neopentyl glycol-adipic acid polyester, PET 4200, PET CS 725, etc.), and/or other ingredients (e.g., water, etc.). In an example where a starting raw material is an oligomer, polymer prediction system 102 calculates descriptors of the oligomer from descriptors of starting raw materials of the oligomer. For example, the starting raw materials of an oligomer can be monomers. As an example, descriptors of a monomer may include sample counts (e.g., a number of carbon atoms, a number of hydrogen atoms, a number of sulfur atoms, a number of oxygen atoms, a number of nitrogen atoms, a number of rotating bonds, a number of carbonyls, a number of double bonds, etc.), percentage weights of sample counts, and/or more sophisticated molecular mechanics and quantum mechanical descriptors. To calculate descriptors of oligomeric starting raw materials, polymer prediction system 102 uses a formula including a weight composition of the monomers in the oligomer and a chemical reaction mechanism or function that produces the oligomers. As another example, polymer prediction system 102 may determine descriptors of the oligomers by applying one or more stoichiometric and mass balance functions or rules and/or one or more probabilistic functions or rules to the descriptors of the monomers. As yet another example, polymer prediction system 102 may determine descriptors of the polymer by applying one or more chemical reaction functions or rules defining the chemical reaction between the ingredients that produce the oligomers and/or the polymer to descriptors of the monomers and/or descriptors of the oligomers and the formula thereof.

In one example, polymer prediction system 102 can receive from, for example, a user an indication that a target polymer includes, as raw materials, 65.33 grams of water, 0.22 grams of DEOA, 1.22 grams of AAS solution, 32 grams of Desmophen PE 225B, 1.11 grams of Kathon LX 1.5%, 0.11 grams of Acetone, and 0.46 grams of Emulgator Tanemul FD 400%. In response, polymer prediction system 102 can retrieve descriptors of these raw materials from descriptor database 104 or, in the case of an oligomer, by first decomposing the oligomer to its raw materials if descriptors of the oligomer are unavailable, and then calculate various descriptors of the target polymer, such as NCO/(OH+NH2) of 1.24, NCO/OH of 1.59, a soft segment percentage of 86.56, a hard segment percentage of 13.44, and/or the like, by applying chemical functions defining chemical reactions between the raw materials to the received descriptors. In some non-limiting embodiments or aspects, polymer prediction system 102 stores the calculated descriptors of the polymer in descriptor database 104 in association with the polymer. Non-limiting examples of descriptors of a target polymer that can be determined according to this process include descriptors from those groups mentioned above. The inventors have found that a variety of descriptors from different areas, in particular, from areas as defined by those descriptor groups described above, when used in combination provide a better prediction of physical properties, including performance properties or characteristics, of polymers and, in particular, a much better prediction of physical properties, including performance properties or characteristics, of PUDs.

In some non-limiting embodiments or aspects, descriptor database 104 also stores descriptor data including a plurality of descriptors of a plurality of polymers other than the target polymer (e.g., a plurality of pre-existing polymers). For example, descriptor database 104 can store descriptors (e.g., calculated descriptors, measured descriptors, predicted descriptors, etc.) for various materials. As an example, descriptor database 104 can store raw material properties (e.g., descriptors of raw materials in the polymer, etc.) in association with functions or calculations applied to the raw material properties that can be used to determine the descriptors of a polymer that includes such raw materials. In some non-limiting embodiments or aspects, polymer prediction system 102 receives, retrieves, modifies, and/or updates the descriptor data in descriptor database 104.

As further shown in FIG. 4, at step 404, process 400 includes determining a prediction of one or more physical properties of a polymer using a predictive model. For example, polymer prediction system 102 determines a prediction of one or more physical properties of a polymer using a predictive model. As an example, polymer prediction system 102 inputs a formula of a target polymer including descriptors of raw materials in the polymer and percentage weights of the raw materials in the polymer to one or more predictive models configured to predict one or more physical properties of the target polymer as described herein, and receives as output a prediction (e.g., a probability, a binary output, a yes-no output, a score, a prediction score, etc.) as to the one or more physical properties (e.g., performance characteristics, etc.) of the target polymer (e.g., as to whether the target polymer includes a physical property or performance characteristic, as to a quantitative value of the physical property or performance characteristic, etc.).

In some non-limiting embodiments or aspects, polymer prediction system 102 determines one or more prediction scores for the target polymer based on the at least one descriptor of the target polymer from the at least three of the descriptor groups as described herein and descriptor data including a plurality of descriptors of a plurality of pre-existing polymers. For example, the descriptor data including a plurality of descriptors of a plurality of pre-existing polymers that are associated with one or more physical properties of the plurality of pre-existing polymers. As an example, polymer prediction system 102 determines one or more prediction scores for a target polymer based on descriptor data and each of the descriptors of the target polymer (or a subset of the descriptors of the target polymer) selected from each of the descriptor groups (or a subset of the descriptor groups). For example, polymer prediction system determines one or more prediction scores for a target polymer based on the descriptor data and selected descriptors of the polymer, where the selected descriptors include at least 3 descriptors, such as, by way of non-limiting examples, at least 15 descriptors, at least 20 descriptors, or at least 50 descriptors.

In some non-limiting embodiments or aspects, polymer prediction system 102 determines one or more prediction scores for a target polymer by also using at least one of the following additional descriptors of the target polymer: an infrared spectra of the polymer, a Raman spectra of the polymer, a particle size distribution of the polymer, or any combination thereof. For example, polymer prediction system 102 can receive an IR spectra, a Raman spectra, and/or a particle size distribution of the target polymer and compare the IR spectra, the Raman spectra, and/or the particle size distribution of the polymer to a library of IR spectra, Raman spectra, and/or particle size distributions for a plurality of polymers. As an example, an IR spectra of a polymer may include absorptions for multiple wave frequencies (e.g., 2000 wave frequencies, etc.), and each wave frequency may be a descriptor of the polymer. As an example, a Raman spectra of a polymer may include shifts for multiple wave frequencies, and each wave frequency may be a descriptor of the polymer. As an example, a particle size distribution of a polymer may include a particle count for multiple size ranges (e.g., 50 size ranges, etc.), and each size range may be a descriptor of the polymer.

In some non-limiting embodiments or aspects, polymer prediction system 102 compares one or more prediction scores of a polymer to one or more threshold values of one or more prediction scores. For example, polymer prediction system 102 determines that a polymer includes one or more physical properties or performance characteristics (e.g., a percentage elongation of the polymer, an adhesion of the polymer to another material, etc.) based on the prediction score of the polymer satisfying the one or more threshold values of the one or more prediction scores. As an example, polymer prediction system 102 generates a polymer (e.g., the target polymer, a polymer structure, a polymer composition, one or more descriptors of a polymer, one or more physical properties of a polymer, etc.) based on a prediction score generated using a machine learning technique by converting the prediction score of the polymer to a value of a descriptor of the polymer by comparing the prediction score to one or more threshold values of the prediction score. For example, polymer prediction system 102 assigns a value (e.g., 1 or 0, a quantitative value or percentage, etc.) to the descriptor of the polymer based on the prediction score of the polymer satisfying the one or more threshold values.

In some non-limiting embodiments or aspects, polymer prediction system 102 determines that a polymer is substantially more similar (or substantially more dissimilar) to one or more polymers than one or more other polymers with respect to one or more physical properties based on one or more prediction scores of the polymer satisfying one or more threshold values of one or more prediction scores. In some non-limiting embodiments or aspects, polymer prediction system 102 generates a representation (e.g., display data for a visual display, such as a heat map, a radar chart, a scatter plot matrix, etc.) of relationships between polymers with respect to the one or more physical properties. For example, polymer prediction system 102 can provide a comparison between polymers with respect to one or more descriptors of the polymers.

As further shown in FIG. 4, at step 406, process 400 includes providing physical property data associated with the target polymer, wherein the physical property data is based on the one or more prediction scores. For example, polymer prediction system 102 provides physical property data (e.g., values of one or more physical properties, display data including values of one or more physical properties, etc.) associated with the target polymer, and the physical property data is based on the one or more prediction scores. As an example, polymer prediction system 102 provides an indication of as to whether the target polymer includes a physical property, such as a performance characteristic, a quantitative value of a descriptor of the physical property for the target polymer, and/or the like.

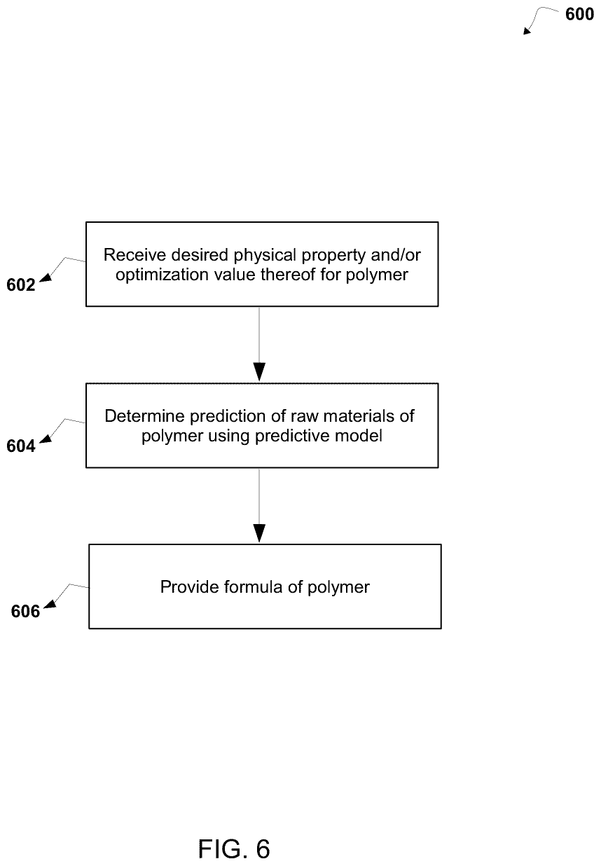

Referring now to FIG. 6, FIG. 6 is a flowchart of a non-limiting embodiment or aspect of a process 600 for predicting a polymer. In some non-limiting embodiments or aspects, one or more of the steps of process 600 may be performed (e.g., completely, partially, etc.) by polymer prediction system 102 (e.g., one or more devices of polymer prediction system 102). In some non-limiting embodiments or aspects, one or more of the steps of process 600 may be performed (e.g., completely, partially, etc.) by another device or a group of devices separate from or including polymer prediction system 102.

As shown in FIG. 6, at step 602, process 600 includes receiving at least one desired physical property of a polymer and/or at least one optimization value of the at least one desired physical property of a polymer. For example, polymer prediction system 102 receives at least one desired physical property of a polymer and/or at least one optimization value of the at least one desired physical property of the polymer. As an example, polymer prediction system 102 receives at least one desired physical property, such as a performance characteristic, of a target polymer and/or at least one optimization value (e.g., a weight associated with the at least one desired physical property, etc.) from user input and/or descriptor database 104.

In some non-limiting embodiments, the at least one desired physical property includes two or more desired physical properties of a target polymer. In such an implementation, a first desired physical property may be assigned a first importance factor, and a second desired physical property may be assigned a second importance factor different than the first importance factor. For example, a value of a physical property or performance characteristic of the target polymer (e.g., tensile strength, etc.) may be weighted with respect to one or more other physicals properties or performance characteristics thereof (e.g., cost, smoothness, etc.) for determining a formula for that target polymer that optimizes the two or more desired physical properties of the target polymer according to the respective weightings or importance factors thereof.

As further shown in FIG. 6, at step 604, process 600 includes determining a prediction of raw materials in a polymer using a predictive model. For example, polymer prediction system 102 determines a prediction of one or more raw materials of a polymer using a predictive model. As an example, polymer prediction system 102 inputs physical property data including at least one desired physical property of a target polymer (and/or at least one optimization value thereof, etc.) to one or more predictive models configured to predict descriptors and/or raw materials of the target polymer as described herein, and receives as output a prediction (e.g., a probability, a binary output, a yes-no output, a score, a prediction score, etc.) as to the one or more descriptors or raw materials of the target polymer (e.g., as to whether the target polymer includes the raw materials, as to a quantitative value of the descriptors of the raw materials, etc.).

In some non-limiting embodiments or aspects, polymer prediction system 102 determines one or more prediction scores for the target polymer based on the physical property data and at least one descriptor of a plurality of pre-existing polymers from at least three of the descriptor groups as described herein. For example, polymer prediction system 102 determines the one or more prediction scores for the target polymer based on at least one descriptor a plurality of pre-existing descriptors from at least three of the following descriptor groups: an atomic species count descriptor group, a functional count descriptor group, a monomer species count descriptor group, an ingredient count descriptor group, a bond type count descriptor group, an atomic species percentage weight descriptor group, a functional percentage weight descriptor group, a monomer species percentage weight descriptor group, an ingredient percentage weight descriptor group, a morphology percentage weight descriptor group, and a polymer parameter and stoichiometry descriptor group. It has been discovered that there is a correlation between descriptors from the above-noted descriptor groups and the ability to predict physical properties, including performance properties or characteristics, of a target polymer through the process described herein. For example, each descriptor, on its own, may be a weak predictor of physical properties, including performance properties or characteristics, of polymers; however, it has been found that the descriptors and descriptor groups discussed herein, when descriptors from at least three different descriptor groups are used in combination, act collectively as a much stronger predictor of physical properties, including performance properties or characteristics, of polymers.

In such an example, the at least one descriptor of the plurality of pre-existing polymers from the at least three of the descriptor groups can be associated with the at least one desired physical property for the plurality of pre-existing polymers. As an example, polymer prediction system 102 determines one or more prediction scores for a polymer based on the physical property data and each of the descriptors of the plurality of pre-existing polymers (or a subset of the descriptors of the plurality of pre-existing polymers) selected from each of the descriptor groups (or a subset of the descriptor groups). For example, polymer prediction system determines one or more prediction scores for a target polymer based on the physical property data and selected descriptors of the plurality of pre-existing polymers, where the selected descriptors include at least 3 descriptors, such as, by way of non-limiting examples, at least 15 descriptors, at least 20 descriptors, or at least 50 descriptors.

In some non-limiting embodiments or aspects, polymer prediction system 102 determines a formula including raw materials or components of the target polymer and percentage weight of the raw material or components of the target polymer based on the desired physical property of the polymer and the descriptors of the pre-existing polymers. For example, polymer prediction system 102 determines raw materials or components in a formula for the polymer based on the desired physical property of the polymer and the descriptors of the pre-existing polymers. As an example, polymer prediction system 102 generates one or more predictions (e.g., one or more prediction scores, etc.) of one or more raw materials (e.g., a concentration of molecular and polymer entities in the polymer, a stoichiometric ratio of reactive groups in a polymerization process for the polymer, etc.) based on a machine learning technique. For example, polymer prediction system 102 generates a model (e.g., an estimator, a classifier, a prediction model, etc.) based on a machine learning algorithm (e.g., a decision tree algorithm, a gradient boosted decision tree algorithm, a neural network algorithm, a convolutional neural network algorithm, a random forest algorithm, etc.) as described herein, and polymer prediction system 102 generates the one or more prediction scores of one or more raw materials in a polymer having the desired physical property or performance characteristic using the model.

Polymer prediction system 102 can use any of many methods to determine a formula of a polymer that provides the at least one desired physical property. For example, polymer prediction system 102 may use a grid method, in which polymer prediction system 102 creates a data base including a full-factorial grid of all possible combinations of ingredients and calculates descriptors for all possible combinations of ingredients, and using the calculated descriptors for all possible combinations of ingredients calculates physical properties for all possible combinations of ingredients, creating in the process a linked database of formulas of polymers, descriptors of the formulas, and predicted physical properties of the polymers defined by the formulas. Polymer prediction system 102 identifies a formula in the linked database that is predicted to have the desired physical properties to determine and provide the formula of a polymer having the desired physical property. Other methods that polymer prediction system 102 may use to determine a formula include, for example, numerical search routines, such as the Nelder-Mead simplex method, the simulated annealing method, and/or the like.

As further shown in FIG. 6, at step 606, process 600 includes providing the formula of the polymer. For example, polymer prediction system 102 provides the formula of the polymer. As an example, polymer prediction system 102 provides descriptors of raw materials, molecular, and/or polymer entities in the target polymer and a stoichiometric ratio of reactive groups in a polymerization process of the target polymer that provides the at least one desired physical property or performance characteristic.

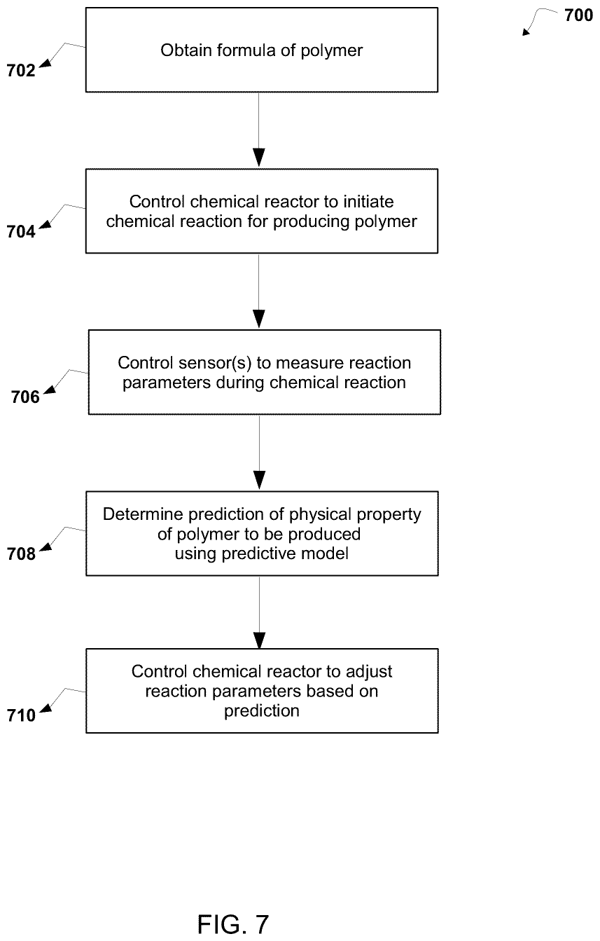

Referring now to FIG. 7, FIG. 7 is a flowchart of a non-limiting embodiment or aspect of a process 700 for predicting a polymer. In some non-limiting embodiments or aspects, one or more of the steps of process 700 may be performed (e.g., completely, partially, etc.) by polymer prediction system 102 (e.g., one or more devices of polymer prediction system 102). In some non-limiting embodiments or aspects, one or more of the steps of process 700 may be performed (e.g., completely, partially, etc.) by another device or a group of devices separate from or including polymer prediction system 102, such as chemical reactor system 108 (e.g., one or more devices of chemical reactor system 108).

As shown in FIG. 7, at step 702, process 700 includes obtaining a formula of a polymer. For example, polymer prediction system 102 obtains a formula of a polymer. As an example, polymer prediction system 102 obtains a formula including a plurality of raw materials for producing a polymer in a chemical reaction.

In some non-limiting embodiments or aspects, a formula of a polymer is associated with one or more desired or target physical properties of a polymer. For example, a formula of a polymer may be associated with one or more desired or target physical properties of a material and/or a product used, produced, and/or existing during a chemical reaction for producing the polymer and/or one or more desired or target physical properties the polymer produced by the chemical reaction. As further shown in FIG. 7, at step 704, process 700 includes controlling a chemical reactor to initiate a chemical reaction for producing a polymer. For example, polymer prediction system 102 controls a chemical reactor to initiate a chemical reaction for producing a polymer. As an example, polymer prediction system 102 controls a chemical reactor (e.g., the chemical reactor of chemical reactor system 108, etc.) to initiate the chemical reaction for producing the polymer of the formula including the plurality of raw materials.

As further shown in FIG. 7, at step 706, process 700 includes controlling one or more sensors to measure one or more reaction parameters during a chemical reaction. For example, polymer prediction system 102 controls one or more sensors to measure one or more reaction parameters during a chemical reaction. As an example, polymer prediction system 102 controls one or more sensors (e.g., the one or more sensors of chemical reactor system 108) to measure one or more reaction parameters during the chemical reaction for producing the polymer. In such an example, the one or more reaction parameters may include at least one of a residence time of a material of the chemical reaction, a volume of a material of the chemical reaction, a volume of an enclosed volume of the chemical reactor for the chemical reaction, a temperature of a material of the chemical reaction, a temperature of a component (e.g., the enclosed volume, a heater, etc.) of the chemical reactor for the chemical reaction, a pressure of material of the chemical reaction, a pressure within an enclosed volume of the chemical reactor for the chemical reaction, concentrations and/or amounts of materials in the chemical reaction, and/or the like.

As further shown in FIG. 7, at step 708, process 700 includes determining a prediction of one or more physical properties of a polymer using a predictive model. For example, polymer prediction system 102 determines a prediction of one or more physical properties of a polymer using a predictive model. As an example, polymer prediction system 102 determines, using a predictive model, one or more prediction scores for the polymer to be produced by the chemical reaction based on the one or more reaction parameters measured during the chemical reaction, wherein the one or more prediction scores include a prediction of one or more physical properties for the polymer to be produced by the chemical reaction.

In some non-limiting embodiments or aspects, polymer prediction system 102 inputs the formula of the polymer to be produced in the chemical reaction (e.g., descriptors of the raw materials for the polymer to be produced and percentage weights for the raw materials in the polymer) and the one or more reaction parameters measured during the chemical reaction to one or more predictive models configured to predict one or more physical properties of the polymer being produced by the chemical reaction as described herein, and receives as output a prediction (e.g., a probability, a binary output, a yes-no output, a score, a prediction score, etc.) as to the one or more physical properties (e.g., performance characteristics, etc.) of the polymer being produced by the chemical reaction (e.g., as to whether the polymer being produced will include a physical property or performance characteristic, as to a quantitative value of the physical property or performance characteristic of the polymer being produced, etc.).

Polymer prediction system 102 can use any of many methods to determine one or more physical properties that are provided by reaction parameters for a formula of a polymer. For example, polymer prediction system 102 may use a grid method, in which polymer prediction system 102 creates a database including a full-factorial grid of all possible combinations of reaction parameters for the formula of the polymer and calculates physical properties for all possible combinations of reaction parameters for the formula of the polymer, creating in the process a linked database of reaction parameters (e.g., sets of reactions parameter) for the formula of the polymer and predicted physical properties for the formula of the polymer for of the reaction parameters (e.g., for the sets of reaction parameters). Polymer prediction system 102 identifies one or more physical properties in the linked database that is predicted to be produced by the one or more reaction parameters for the formula of polymer to determine the prediction of the one or more physical properties for the polymer to be produced by the chemical reaction. Other methods that polymer prediction system 102 may use to determine the one or more physical properties include, for example, numerical search routines, such as the Nelder-Mead simplex method, the simulated annealing method, and/or the like.

As further shown in FIG. 7, at step 710, process 700 includes controlling a chemical reactor to adjust one or more reaction parameters based on a prediction of one or more physical properties of a polymer using a predictive model. For example, polymer prediction system 102 controls a chemical reactor to adjust one or more reaction parameters based on a prediction of one or more physical properties of a polymer using a predictive model. As an example, polymer prediction system 102 controls, during the chemical reaction for producing the polymer, the chemical reactor (e.g., the one or more control devices of the chemical reactor of chemical reactor system 108) to adjust the one or more reaction parameters based on the one or more prediction scores.

Polymer prediction system 102 can use any of many methods to determine one or more reaction parameters for producing a polymer that provides the at least one desired or targeted physical property associated with the formula of the polymer. For example, polymer prediction system 102 may use the grid method described herein above to identify the one or more reaction parameters (e.g., the set of reaction parameters, etc.) in the linked database that are predicted to produce the polymer having the desired or targeted physical properties to determine an adjustment (e.g., an amount of change, a difference to implement, etc.) to the one or more reaction parameters during the chemical reaction that is based on the one or more prediction scores. Other methods that polymer prediction system 102 may use to determine reaction parameters include, for example, numerical search routines, such as the Nelder-Mead simplex method, the simulated annealing method, and/or the like.

In some non-limiting embodiments or aspects, polymer prediction system 102 compares the one or more measured reaction parameters to the one or more reaction parameters determined as producing to one or more desired or target physical properties associated with the formula for producing the polymer. For example, polymer prediction system 102, using the predictive model that predicts the one or more physical properties of the polymer to be produced as a function of the measured reaction parameters for the formula of the polymer, may determine the one or more reaction parameters to be adjusted and an amount of adjustment to be made to the one or more reaction parameters to cause the prediction of the one or more physical properties of the polymer to be produced to converge toward the one or more desired or target physical properties associated with the formula for producing the polymer. As an example, polymer prediction system 102 may adjust the one or more reaction parameters to minimize or reduce a difference (e.g., a difference in values) between the one or more physical properties of the polymer to be produced and the one or more desired or target physical properties associated with the formula for producing the polymer.

The foregoing disclosure provides illustration and description, but is not intended to be exhaustive or to limit the implementations to the precise form disclosed. Modifications and variations are possible in light of the above disclosure or may be acquired from practice of the implementations.

Some implementations are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

It will be apparent that systems and/or methods, described herein, can be implemented in different forms of hardware, software, or a combination of hardware and software. The actual specialized control hardware or software code used to implement these systems and/or methods is not limiting of the implementations. Thus, the operation and behavior of the systems and/or methods are described herein without reference to specific software code it being understood that software and hardware can be designed to implement the systems and/or methods based on the description herein.

Even though particular combinations of features are recited in the claims and/or disclosed in the specification, these combinations are not intended to limit the disclosure of possible implementations. In fact, many of these features can be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Furthermore, as used herein, the term “set” is intended to include one or more items (e.g., related items, unrelated items, a combination of related and unrelated items, etc.), and may be used interchangeably with “one or more.” Where only one item is intended, the term “one” or similar language is used. Also, as used herein, the terms “has,” “have,” “having,” and/or the like are intended to be open-ended terms. Further, the phrase “based on” is intended to mean “based, at least in part, on” unless explicitly stated otherwise.

1-28. (canceled)

29. A method for producing a polymer, comprising:
obtaining, with a computer system comprising one or more processors, a formula including a plurality of raw materials for producing a polymer in a chemical reaction;
obtaining, with the computer system, one or more desired or target physical properties the polymer produced by the chemical reaction;
controlling, with the computer system, a chemical reactor to initiate the chemical reaction for producing the polymer;
measuring one or more reaction parameters during the chemical reaction;
determining, with the computer system, one or more prediction scores for the polymer to be produced by the chemical reaction based on the one or more reaction parameters measured during the chemical reaction, wherein the one or more prediction scores include a prediction of one or more physical properties for the polymer to be produced; and
during the chemical reaction, controlling, with the computer system, the chemical reactor to adjust the one or more reaction parameters based on the one or more prediction scores.

30. The method of claim 29, wherein the one or more desired or target physical properties of the polymer include at least one of the following: a tensile strength, an adhesion, an elongation, a hardness, a viscosity of the polymer in water, a cost associated with the polymer, a mean percentage of solids, a minimum flow rate, a maximum flow rate, a minimum viscosity, a maximum viscosity, a pH level, a number of acids from RS, a number of OH #, a particle size, a modulus at 100%, an ultimate strength, an elongation percentage, a softening point, a heat activation temperature, a glass transition temperature, a microhardness, a pendulum hardness, a chemical residue of H2SO4, a chemical residue of NaOH, a chemical residue of ethanol, a chemical residue of water, a chemical residue of xylene, a hydrolytic stability, a dry time, an amount of Mn, an amount of Mw, a direct impact strength, an inverse impact strength, a Taber abrasion, a Taber comment, a gloss level, results of a cross hatch test, or any combination thereof.

31. The method of claim 29, wherein the one or more reaction parameters includes at least one of a residence time of a material of the chemical reaction, a volume of a material of the chemical reaction, a volume of an enclosed volume of the chemical reactor for the chemical reaction, a temperature of a material of the chemical reaction, a temperature of a component of the chemical reactor for the chemical reaction, a pressure of material of the chemical reaction, a pressure within an enclosed volume of the chemical reactor for the chemical reaction, concentrations or amounts of materials in the chemical reaction.

32. The method of claim 29, further comprising:
comparing the one or more measured reaction parameters to the one or more reaction parameters determined to be produced according to one or more desired or target physical properties associated with the formula for producing the polymer;
determining if one or more reaction parameters need to be adjusted and an amount of adjustment to be made to the one or more reaction parameters to cause the prediction of the one or more physical properties of the polymer to be produced to converge toward the one or more desired or target physical properties; and
adjusting one or more reaction parameters to minimize or reduce a difference between the one or more physical properties of the polymer to be produced and the one or more desired or target physical properties associated with the formula for producing the polymer.

33. The method of claim 29, further comprising:
receiving, with the computer system, at least two desired physical properties of the target polymer, wherein a first desired physical property is assigned a first importance factor, wherein a second desired physical property is assigned a second importance factor different than the first importance factor; and
determining, with the computer system, the one or more prediction scores for the target polymer based on the at least two desired physical properties of the target polymer, the first importance factor, and the second importance factor.
